(19) United States
(12) Patent Application Publication
(10) Pub. No.: US 2008/0255168 A1
(43) Pub. Date: Oct. 16, 2008

(54) METHODS FOR ADMINISTERING DPD INHIBITORS IN COMBINATION WITH 5-FU AND 5-FU PRODRUGS

(75) Inventors: William Paul Peters, Fernandina Beach, FL (US); Mukur Gupta, Morrisville, NC (US)

Correspondence Address: SEED INTELLECTUAL PROPERTY LAW GROUP PLLC, 701 FIFTH AVE, SUITE 5400, SEATTLE, WA, 98104, US

(73) Assignee: ADHEREX TECHNOLOGIES, INC., Ottawa (CA)

(21) Appl. No.: 12/027,064

(22) Filed: Feb. 6, 2008

Related U.S. Application Data
(63) Continuation-in-part of application No. 11/294,643, filed on Dec. 5, 2005.
(60) Provisional application No. 60/633,034, filed on Dec. 3, 2004.

(51) Int. Cl. A61K 31/505; A61P 17/00

(52) U.S. Cl. 514/274

(57) ABSTRACT

Methods for improved administration and dosing of DPD inhibitors in combination with 5-FU and/or 5-FU prodrugs are provided, comprising first administering to a patient in need thereof a DPD inhibitor that substantially eliminates activity of the enzyme and thereafter administering 5-FU or a 5-FU prodrug, wherein the level of 5-FU or 5-FU prodrug is in substantial excess of DPD inhibitor in the patient. Also provided are topical formulations comprising DPD, TP and/or UP inhibitors and methods of using same.

### CROSS-REFERENCE TO RELATED APPLICATIONS

This application is a continuation-in-part of U.S. patent application Ser. No. 11/294,643, filed Dec. 5, 2005, now pending, which application claims the benefit under 35 U.S.C. § 119(e) of U.S. Provisional Patent Application No. 60/633,034, filed Dec. 3, 2004, wherein these applications are incorporated herein by reference in their entireties.

### BACKGROUND OF THE INVENTION

1. Field of the Invention

This invention relates generally to cancer therapy, and more particularly to cancer therapy using DPD inhibitors in combination with 5-FU and/or 5-FU prodrugs.

2. Description of the Related Art

5-Fluorouracil (5-FU) has been clinically used to treat solid tumors in cancer patients for over three decades (Ansfield et al., Cancer 39: 34-40, 1977; Grem et al., Cancer Treat Rep 71: 1249-1264, 1987; Chabner et al., Cancer, Principles and Practice of Oncology, 2nd Ed, pp 287-328 Philadelphia, Pa.: J B Lippincott Co, 1985). 5-FU must be activated by metabolic conversion to fraudulent uridine nucleotides (e.g., FUMP, FUDP, FUTP) and deoxyuridine nucleotides (e.g., FdUMP, FdUDP, FdUTP) that interfere with DNA synthesis and RNA functions (reviewed in Meyers, Pharmacol Rev, 33: 1-15, 1981; Dasher et al., Pharmac Ther 48: 189-222, 1990). Because 5-FU differs from uracil, its natural counterpart, by only a fluorine substitution in the 5-position, it is readily activated in cancer patients. Unfortunately, its structural similarity to uracil also accounts for its rapid and extensive conversion to breakdown products that have no antitumor activity. This metabolic process is referred to as inactivation. 5-FU is rapidly inactivated by the enzyme dihydropyrimidine dehydrogenase (DPD: EC 1312, uracil reductase) (Meyers, Pharmacol Rev, 33: 1-15, 1981; Dasher et al., Pharmac Ther 48: 189-222, 1990). Therefore, the antitumor efficacy of 5-FU for treating cancer relies on the delicate balance between metabolic conversion to antitumor nucleotides (activation) and metabolic conversion to useless metabolites (inactivation).

Furthermore, several clinical issues arise due to the metabolic inactivation of 5-FU. First, because the levels of DPD vary among individuals (Fleming et al., Cancer Res 52: 2899-2902, 1992; Grem et al., Cancer Chemother Pharmacol 40: 117-125, 1997) and within individuals during the course of a day (Grem et al., Cancer Chemother Pharmacol 40: 117-125, 1997; Harris et al., Cancer Res 50: 197-201, 1990; Petit et al., Cancer Res 48: 1676-1679, 1988), the systemic levels of 5-FU produced from a given dose vary greatly and therefore render efficacy and toxicity highly unpredictable. At the extreme, patients genetically deficient in DPD experience severe and sometimes fatal toxicity when treated with ‘standard’ therapeutic doses of 5-FU (reviewed in Morrison et al., Oncol Nurs Forum 24: 83-88, 1997). Second, variable levels of gastrointestinal DPD (Ho et al., Anticancer Res 6: 781-784, 1986; Naguib et al., Cancer Res 45: 5405-5412, 1985; Spector et al., Biochem Pharmacol 46: 2243-2248, 1993) create highly variable absorption of orally dosed 5-FU (Christophidis et al., Clin Pharmacokinetics 3: 330-336, 1978; Cohen et al., Cancer Chemother Rep 58: 723-731, 1974; Finch et al., Br J Clin Pharmacol 7: 613-617, 1979) and therefore can result in unpredictable plasma levels of drug and produce undesirable toxicity or inadequate efficacy. Third, tumors containing high levels of DPD are less likely to respond to 5-FU-treatment (Etienne et al., J Clin Oncol 13: 1663-1670, 1995; Fischel et al., Clin Cancer Res 1: 991-996, 1995). Finally, the breakdown products of 5-FU may produce neurotoxicity (Okeda et al., Acta Neuropathol 81: 66-73, 1990; Koenig et al. Arch Neurol 23: 155-160, 1970), cardiotoxicity (et al., Lancet 337: 560, 1991; Lemaire et al., Br J Cancer 66: 119-127, 1992), palmer-plantarerythrodysaesthesia (hand-foot syndrome) (Hohneker, Oncology 12: 52-56, 1998), and GI toxicity (Spector et al., Cancer Res 55: 1239-1241, 1995) and appear to interfere with the antitumor activity (Spector et al., Cancer Res 55: 1239-1241, 1995; Cao, et al., Pharmacol 59: 953-960, 2000).

DPD is a ubiquitous enzyme that is the first and the rate-limiting step in the degradation (inactivation) of 5-FU. Studies have shown that inhibition of DPD prolongs the half-life of 5-FU in plasma. Several DPD inhibitors have been studied, including those that irreversibly inactivate DPD as well as those that reversibly inhibit DPD.

5-Ethynyluracil, also referred to as eniluracil, is an example of a DPD inhibitor that is an irreversible inactivator of DPD that reduces or eliminates the metabolic inactivation of 5-FU (for reviews see Spector et al., Drugs of The Future 19: 565-571, 1994; Paff et al., Invest. New Drugs: 18, 365-371 (2000)). Due to the structural similarity between eniluracil and 5-FU, eniluracil is a substrate for DPD. As DPD attempts to break down eniluracil, the latter is converted to a highly reactive compound that irreversibly binds to DPD and thereby inactivates the enzyme. Thus, in the presence of very low amounts of eniluracil, DPD is destroyed and is no longer capable of inactivating 5-FU. Active DPD only reappears in such patients as a result of de novo DPD enzyme synthesis over a course of days.

Eniluracil has been tested in Phase I clinical trials in cancer patients (reviewed in Levin et al., Invest New Drugs 18:383-90, 2000; Baker et al., J Clin Oncol 18: 915-926 2000; Schilsky et al., J Clin Oncol 4:1450-7, 1998). It very potently eliminated DPD activity without causing toxicity. A dose of 0.74 mg/m² (about 1 mg total) eliminated greater than 90% of all DPD for prolonged periods. In fact, 24 hours after one dose of eniluracil, the level of DPD was only 3% of the predose level. The elimination half-life of 5-FU was increased from about 10 minutes to 3.5 hours by one dose of eniluracil. A dose of 3.7 mg/m² eniluracil increased the half-life of 5-FU to 4.5 hours. Higher doses added no apparent benefit.

Eniluracil has also been orally administered in Phase II and Phase III clinical trials (reviewed in Levin et al., Invest New Drugs 18:383-90, 2000; Schilsky et al., J Clin Oncol: 20:1519-26, 2002). Two dosing regimens were used in these trials. In the ‘5-day schedule’, eniluracil was administered at a fixed dose of 50 mg per day on day-1 through day-7. 5-FU was dosed at approximately 20 mg/m² on day-2 through day-6 after the dose of eniluracil. In the ‘28-day schedule’, eniluracil and 5-FU were co-administered in a fixed ratio of ten eniluracil to one 5-FU b.i.d. (twice daily) for 28 days. The dose of 5-FU was approximately 1 mg/m². Eniluracil abrogated the 5-FU-associated hand-foot syndrome toxicity, enabled 5-FU to be safely dosed orally, and resulted in highly predictable 5-FU plasma levels. However, the antitumor activity of these regimens was unfortunately disappointing. In two multicenter Phase III studies using the 28-day eniluracil regimen for colorectal cancer, patients receiving eniluracil and 5-FU tended to have less antitumor activity than those treated with the standard 5-FU regimen without eniluracil (Schilsky et al., J Clin Oncol: 20:1519-26, 2002).

Hand-foot syndrome (HFS) is a well described, cumulative, dose limiting toxicity of certain commonly utilized cancer chemotherapy agents, particularly the fluororopyrimidines. Symptoms typically occur within the first few cycles of therapy and initially include numbness and tingling in the hands and feet. This is followed by plamar and plantar erythema, with subsequent blistering. The nature of this toxicity results in substantial patient discomfort and delays in treatment. Drugs most frequently implicated in causing HFS include 5-fluorouracil, liposomal doxorubicin (Doxil), cytarabine, docetaxel and 5-FU prodrugs, such as capecitabine (Xeloda®), and tegafur.

The cause of HFS following treatment with 5-FU and 5-FU prodrugs treatment appears to result from catabolic products of 5-FU, most likely FBAL, which are produced by the metabolism of 5-FU in skin tissues. However, some data also suggest that capecitabine induced HFS results from excess activation of capecitabine in keratinocytes via thymidine phosphorylase (Fischel 2004). Thus, inhibition of the conversion of the 5-FU prodrug to 5-FU via inhibition of thymidine phosphorylase (TP) or uridine phosphorylase (UP), or catabolism of the 5-FU via inhibition of DPD, offer potential ways of interrupting the development of HFS.

The mechanism of HFS is unclear and despite trials using various topical agents, there is currently no established preventative or therapeutic strategy for effectively addressing this condition (Gressett 2006). The typical approach to a patient who has experienced HFS is to wait for the symptoms to resolve to grade 1 and then reduce the dose of the suspected chemotherapy drug for subsequent cycles.

Thus, there remains an important and unmet need in the art for identifying optimal dosing and administration schedules for DPD inhibitors used in combination with 5-FU and 5-FU prodrugs in order to maximize the antitumor efficacy and therapeutic index of 5-FU and 5-FU prodrugs, to improve the predictability of dosing and to enable 5-FU and 5-FU prodrugs to be effectively dosed by oral administration. There also remains a need for identifying optimal formulations and administration approaches for DPD, TP and/or UP inhibitors used in combination with 5-FU and 5-FU prodrugs in order to reduce the frequency and/or severity of HFS.

The present invention fulfills these needs and offers other related advantages.

### SUMMARY OF THE INVENTION

The present invention relates generally to improved methods for administering DPD inhibitors, such as eniluracil, in combination with 5-FU and 5-FU prodrugs. Therefore, according to one aspect of the present invention, there is provided a method for treating cancer in a patient comprising first administering a DPD inhibitor that substantially eliminates this enzyme and thereafter administering 5-FU or a 5-FU prodrug, wherein the 5-FU or 5-FU prodrug is administered at a dose such that at its time of administration the 5-FU or 5-FU prodrug is present in the patient in substantial excess of the free DPD inhibitor.

In one embodiment according to this aspect of the invention, the 5-FU or 5-FU prodrug is administered at least about 4 hours, at least about 6 hours, at least about 8 hours, at least about 10 hours, at least about 12 hours, at least about 14 hours, at least about 24 hours, or at least about 36 hours after the DPD inhibitor is administered.

In another embodiment according to this aspect of the invention, the 5-FU or 5-FU prodrug is administered about 4-72 hours, 4-36 hours, 4-24 hours, 4-14 hours, 6-14 hours or 8-14 hours after the DPD inhibitor is administered.

In another embodiment according to this aspect of the invention, the 5-FU or 5-FU prodrug is administered at a time when at least about 0.1-4 elimination half-lives, 1-4 elimination half-lives, 2-4 elimination half-lives or 3-4 elimination half-lives for the DPD inhibitor have passed since the DPD inhibitor was administered.

In another embodiment according to this aspect of the invention, the DPD inhibitor is administered at a dose sufficient to reduce DPD activity in the patient to less than about 10%, less than about 5%, less than about 3% or less than about 1% of baseline DPD activity in the patient.

In another embodiment according to this aspect of the invention, the 5-FU or 5-FU prodrug is administered at a dose such that at its time of administration the 5-FU or 5-FU prodrug is present in the patient in at least about 2-fold, at least about 3-fold, at least about 5-fold or at least about 100-fold excess of the DPD inhibitor.

In another embodiment according to this aspect of the invention, the DPD inhibitor is an irreversible DPD inhibitor.

In another embodiment according to this aspect of the invention, the DPD inhibitor is a reversible DPD inhibitor. In such embodiments, it will be understood that certain preferred reversible DPD inhibitors include tight-binding inhibitors that dissociate from DPD more slowly than excess inhibitor is eliminated from the body and therefore will not be present in substantial excess when 5-FU or the 5-FU prodrug is administered. Other preferred inhibitors include those that inhibit DPD activity but do not substantially inhibit other enzymes that activate fluorouracil, such as uridine phosphorylase (UPase), orotate phosphoribosyltransferase (OPRTase) and thymidine phosphorylase (TP).

In another embodiment according to this aspect of the invention, the 5-FU or 5-FU prodrug is selected from the group consisting of the following compounds and their 5′-esters, including phosphate esters: 5-fluorouridine, 5-fluorocytidine, 5-fluoro-2-deoxyuridine, 5-fluoro-2-deoxycytidine, and 5-fluoroarabinosyluracil.

In another embodiment according to this aspect of the invention, the 5-FU or 5-FU prodrug is selected from the group consisting of 5′-deoxy-4′,5-fluorouridine, 5′-deoxy-5-fluorouridine, 1-(2-tetrahydrofuranyl)-5-fluorouracil, a 1-C₁₋₈ alkylcarbamoyl-5-fluorouracil derivative, 1-(2-tetrahydrofuryl)-5-fluorouracil, 5′-deoxy-5-fluoro-N-[(pentyloxy)carbonyl]-cytidine (capecitabine), or a compound that is converted to 5-FU in vivo.

In another embodiment according to this aspect of the invention, the DPD inhibitor is eniluracil or a prodrug thereof.

In another embodiment according to this aspect of the invention, the DPD inhibitor is eniluracil, and is administered at a dose of 0.8-15, 2-15, 5-15 or 2.5-5 mg/m².

In another embodiment according to this aspect of the invention, the DPD inhibitor is eniluracil and the 5-FU or 5-FU prodrug is 5-FU.

In another embodiment according to this aspect of the invention, the 5-FU or 5-FU prodrug is 5-FU, and is administered at a dose 0.5-80, 0.5-40, 10-80, 10-60, 10-30 or 20-60 mg/m².

In another embodiment of the invention, the eniluracil and 5-FU or 5-FU prodrug are dosed at a ratio of about 1:3 to 1:20, 1:5 to 1:15, or 1:8 to 1:12.

In one preferred embodiment, eniluracil is first administered at a dose of about 2.5-5 mg/m² at least about 10-14 hours before administering 5-FU or 5-FU prodrug at a dose of about 15-30 mg/m² or before administering capecitabine at a dose of about 5-100 mg/m². Optionally, additional doses of 5-FU or capecitabine can be thereafter administered with or without additional dosing of eniluracil.

In another embodiment according to this aspect of the invention, the DPD inhibitor is eniluracil and the 5-FU or 5-FU prodrug is capecitabine.

In another embodiment according to this aspect of the invention, the DPD inhibitor is eniluracil, the 5-FU or 5-FU prodrug is 5-FU or capecitabine, and the eniluracil is administered at a dose between about 0.8-15, 2.5-15, 5-15 or 2.5-5 mg/m².

In another embodiment according to this aspect of the invention, the DPD inhibitor is eniluracil, the 5-FU or 5-FU prodrug is 5-FU, the eniluracil is administered at a dose between about 2.5-5 mg/m², and the 5-FU is administered at a dose between about 0.5-40 mg/m², depending on the dosing schedule used.

One illustrative schedule, e.g., a twice per day (b.i.d.) for 28 days (28-day) schedule, employs a 5-FU dose from between about 0.5-1.5 mg/m². Another illustrative schedule, e.g., a once per day for 5 days (5-day) schedule, employs a 5-FU dose from between about 10-60 mg/m². In a more particular embodiment, the 5-FU dose is between about 10-30 mg/m². In another embodiment, the 5-FU dose is between about 20-60 mg/m².

Another illustrative schedule, e.g., a once per week (weekly) schedule, can employ a 5-FU dose between about 10-80 mg/m². In a more particular embodiment, 5-FU is dosed between about 15-40 or 10-30 mg/m². In another embodiment, 5-FU is dosed between about 30-80 mg/m².

In another embodiment according to this aspect of the invention, the DPD inhibitor is eniluracil, the 5-FU or 5-FU prodrug is capecitabine, the eniluracil is administered at a dose between about 0.8-15, between about 2-15, between about 5-15 or between about 2.5-5 mg/m², and the capecitabine is administered at a dose between about 0.8-200 mg/m². In a more particular embodiment, the capecitabine is administered at a dose between about 0.8-10 mg/m² or 1.3-4 mg/m² (e.g., for certain illustrative protracted b.i.d. daily schedules).

In another embodiment according to this aspect of the invention, the DPD inhibitor comprises a 5-substituted uracil analog or a prodrug thereof.

In another embodiment according to this aspect of the invention, the DPD inhibitor comprises a uracil analog substituted in the 5-position by a halogen atom, a C₂₋₄ alkenyl group, a C₂₋₄ alkenyl group substituted by halogen, a C₂₋₆ alkynyl group, a C₂₋₆ alkynyl group substituted by a halogen, a cyano group or a C₁₋₄ alkyl group substituted by halogen.

In another embodiment according to this aspect of the invention, the DPD inhibitor comprises a uracil analog selected from the group consisting of eniluracil, 5-propynyluracil, 5-cyanouracil, 5-propynyluracil, 5-bromoethynyluracil, 5-(1-chlorovinyl)uracil, 5-iodouracil, 5-bromovinyluracil, (E)-5-(2-bromovinyl)uracil 5-hex-1-ynyluracil, 5-vinyluracil, 5-trifluorouracil, 5-bromouracil, and 5-(2-bromo-1-chlorovinyl)uracil.

In another embodiment according to this aspect of the invention, the DPD inhibitor is selected from the group consisting of 5-(phenylselenenyl)uracil (PSU), 5-(phenylthio)uracil (PTU), 5-(phenylselenenyl)barbituric acid and 5-(phenylthio)barbituric acid.

According to another aspect of the invention, there is provided a method for treating cancer in a patient comprising first administering eniluracil and thereafter administering 5-FU, wherein the 5-FU is administered at a dose such that at its time of administration the 5-FU is present in the patient in substantial excess of the DPD inhibitor.

In one embodiment according to this aspect of the invention, the 5-FU is administered at least about 4 hours, 6 hours, 8 hours, 10 hours, 12 hours, 14 hours, 24 hours or 36 hours after the eniluracil is administered. In one preferred embodiment, the 5-FU is administered at least about 4 hours, at least about 8 hours or at least about 12 hours after the eniluracil is administered.

In another embodiment according to this aspect of the invention, the 5-FU is administered about 4-72 hours, 4-36 hours, 4-24 hours, 4-14 hours, 6-14 hours or 8-14 hours after the eniluracil is administered. In one preferred embodiment, the 5-FU is administered about 4-14 hours after the eniluracil is administered.

In another embodiment of the invention, the 5-FU is administered at a time when between about 0.1-4 elimination half-lives, 1-4 elimination half-lives, 2-4 elimination half-lives or 3-4 elimination half-lives for the eniluracil have passed since the eniluracil was administered.

In another embodiment according to this aspect of the invention, the eniluracil is administered at a dose sufficient to reduce DPD activity in the patient to less than about 10%, less than about 5%, less than about 3% or less than about 1% of baseline DPD activity.

In another embodiment according to this aspect of the invention, the 5-FU is administered at a dose such that at its time of administration the 5-FU is present in the patient in at least about 2-fold, at least about 3-fold, at least about 5-fold or at least about 100-fold excess of the eniluracil.

In another embodiment according to this aspect of the invention, the eniluracil is administered at a dose between about 0.7-15 mg/m². In another embodiment of the invention, the eniluracil is administered at a dose between about 2.5-5 mg/m². In another embodiment of the invention, the eniluracil is administered at a dose between about 5-15 mg/m².

In another embodiment according to this aspect of the invention, the eniluracil is administered at a dose between about 2.5-5 mg/m² and the 5-FU is administered at a dose between about 0.5-40 mg/m².

According to another aspect of the present invention, there is provided a method for treating cancer in a patient comprising first administering eniluracil and thereafter administering 5-FU, wherein the eniluracil is administered at a dose between about 2.5-5 mg/m², and wherein the 5-FU is administered when between about 1-4, 2-4 or 3-4 elimination half-lives for eniluracil have passed since the eniluracil was administered.

In one embodiment according to this aspect of the invention, the 5-FU is administered at least about 4 hours, 6 hours, 8 hours, 10 hours, 12 hours or 14 hours after the eniluracil is administered.

In one embodiment according to this aspect of the invention, the 5-FU is administered between about 4-36 hours, 4-24 hours, 4-14 hours, 6-14 hours or 8-14 hours after the eniluracil is administered.

In another embodiment according to this aspect of the invention, the 5-FU is administered at a dose such that at its time of administration the 5-FU is present in the patient in at least about 2-fold, at least about 3-fold, at least about 5-fold or at least about 100-fold excess of the eniluracil.

In another embodiment according to this aspect of the invention, the 5-FU is administered at a dose between about 0.5-40 mg/m².

In a more particular embodiment, eniluracil (or another DPD inhibitor) is first administered and then multiple doses of 5-FU or 5-FU prodrug are thereafter administered at desired time points, before eniluracil (or another DPD inhibitor) is optionally administered again. For example, in one preferred embodiment, eniluracil is first administered at about 2.5 to 5 mg/m² at least about 10 to 14 hours before administration of the 5-FU or 5-FU prodrug, for example on the evening prior to the first day that either 5-FU is administered at about 15 to 30 mg/m² or capecitabine at about 5-100 mg/m² and then similar multiple doses of 5-FU or capecitabine are thereafter administered. For example, in one illustrative embodiment, eniluracil is first dosed and then multiple 5-FU or capecitabine doses are administered daily for three days each week, for example, before eniluracil is optionally administered again and the cycle repeated.

According to another aspect of the present invention, there is provided a method for treating cancer in a patient comprising first administering eniluracil and thereafter administering 5-FU, wherein the eniluracil is administered at a dose between about 2.5-5 mg/m², the 5-FU is administered when between 1-4, 2-4 or 3-4 elimination half-lives for eniluracil have passed since the eniluracil was administered, and the 5-FU is administered at a dose between about 0.5-40 mg/m².

According to another aspect of the present invention, there is provided an oral pharmaceutical time-release formulation comprising a DPD inhibitor and 5-FU or a 5-FU prodrug, wherein following administration of the formulation to a patient the 5-FU or 5-FU prodrug is not substantially released until about 0.5-36, 4-36, 4-24 or 4-14 hours after the DPD inhibitor has been released.

According to another aspect of the invention, there is provided a pharmaceutical formulation comprising a DPD inhibitor and a delivery vehicle for administering the DPD inhibitor to a patient. In another embodiment, the formulation comprises a DPD inhibitor and 5-FU or a 5-FU prodrug. In another embodiment, the formulation comprises a delivery vehicle, a DPD inhibitor and 5-FU or a 5-FU prodrug. In a particular embodiment, the delivery vehicle is a microsphere. In a related embodiment, the delivery vehicle is a microsphere that allows for the preferential or selective targeting of the DPD inhibitor to cancer cells using formulation and delivery techniques known in the art.

According to another aspect of the invention, DPD inhibitor administered in accordance with the invention is a topical formulation, and may be administered or applied, for example, to the skin of a patient undergoing therapy with 5-FU or 5-FU prodrug. As demonstrated herein, locally administered formulations comprising DPD inhibitors can effectively block DPD activity in the skin of animals. Further, systemic 5-FU pharmacokinetics and systemic DPD enzyme activity can be modulated to a desired extent by selection of proper dosing and exposure time of the topical formulation.

Therefore, in certain embodiments, the present invention provides topical formulations and methods which can be used for reducing the frequency and/or severity of HFS by proper dosing and administration of DPD inhibitors locally to the skin, e.g., the hands and/or feet, of a patient undergoing treatment with 5-FU or 5-FU prodrug.

In other embodiments, there are provided methods for reducing the frequency and/or severity of Hand-Foot Syndrome (HFS) in a patient undergoing treatment with 5-FU or a 5-FU prodrug, the methods comprising contacting the hands and/or feet of the patient with a topical formulation comprising an effective dose of an irreversible DPD inhibitor. In certain embodiments, such formulations are designed and administered to effectively inhibit DPD activity in the skin, e.g., in the hands and/or feet of a patient, without substantially inhibiting systemic DPD activity in the patient. In other embodiments, such formulations are designed and administered to effectively inhibit DPD activity in the skin, e.g., in the hands and/or feet of a patient, while also achieving a desired level of systemic DPD inhibition in the patient.

The topical formulation can be in any suitable or conventional form, illustrative examples of which include an ointment, cream, lotion, aerosol spray, roll-on liquid, pad form, and the like. In certain embodiments, additional compounds are added which restrict blood flow to the area or by other means reduce systemic absorption of the DPD inhibitor.

In certain embodiments, the concentration of DPD inhibitor present in a topical formulation is a concentration effective to achieve the desired level of local DPD inhibition while not substantially effecting systemic DPD activity. In certain particular embodiments, the concentration of DPD inhibitor in a topical formulation will range from about 0.0.001 to about 0.05 w/w.

Following application, the topical formulation may be optionally removed after a sufficient exposure time has elapsed. For example, in certain embodiments, the topical formulation will be removed or washed from the skin after an exposure time of about 1 to about 60 minutes.

The topical formulation, in certain embodiments, may be applied before, at the same time as, or after 5-FU or 5-FU prodrug treatment, and the topical formulation may be applied one or multiple times during each course of 5-FU or 5-FU prodrug treatment. In certain other embodiments, the topical formulation is applied prior to 5-FU or 5-FU prodrug treatment, for example about 5 min to 72 hours prior to administration of 5-FU or 5-FU prodrug to a patient.

According to another aspect of the invention, there are provided topical formulations for reducing the frequency and/or severity of Hand-Foot Syndrome (HFS) in a patient undergoing treatment with 5-FU or a 5-FU prodrug, the topical formulation comprising an effective dose of an irreversible DPD inhibitor. In certain embodiments, the effective dose of DPD inhibitor in the topical formulation inhibits DPD activity in the hands and/or feet but does not result in systemic DPD inhibition. In a particular embodiment, the concentration of DPD inhibitor in the topical formulation is about 0.001 to about 0.08 w/w. The topical formulation may be in any suitable or convenient form, for example selected from the group consisting of an ointment, cream, lotion, aerosol spray, roll-on liquid and pad form, as further described herein.

These and other aspects of the invention will be apparent upon reference to the following detailed description and attached figures. Patent and other documents cited herein to more specifically set forth various aspects of this invention are hereby incorporated by reference in their entireties.

### BRIEF DESCRIPTION OF THE DRAWINGS

FIG. 1 shows the chemical structures of eniluracil and 5-FU.

FIG. 2 shows that eniluracil causes increased plasma uridine levels following administration in mice.

FIG. 3 shows an illustrative oral time-release formulation comprising eniluracil and 5-FU in a tablet form.

FIG. 4 shows a plasma concentration time profile for 5-FU following in vivo administration.

FIG. 5 shows a plasma concentration time profile for 5-FU following administration of capecitabine in vivo.

FIG. 6 shows that a single administration of an illustrative topical formulation comprising eniluracil had no effect on systemic 5-FU pharmacokinetics in vivo.

FIGS. 7A-D show the pharmacokinetic profile of individual formulations administered in vivo on days 0, 7 and 14.

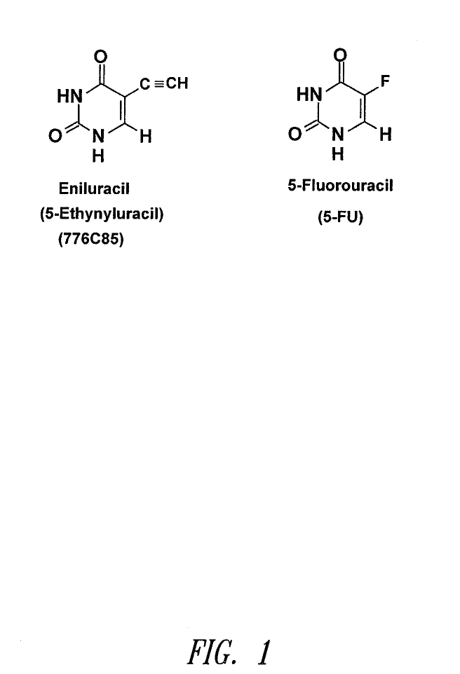

### DETAILED DESCRIPTION OF THE INVENTION

As noted above, the present invention relates generally to methods for treating cancer which comprise administering to a patient at least one DPD inhibitor in combination with at least one 5-FU or 5-FU prodrug, and to compositions and formulations useful in such methods. The methods described herein are thus applicable to the treatment of essentially any cancer type in which 5-FU and/or 5-FU prodrugs have activity, including, by way of illustration but not by way of limitation, breast cancer, lung cancer, colon cancer, pancreatic cancer, gastric cancer, bladder, renal cancer, head and neck cancer, esophageal cancer, hepatocellular cancer, and all malignant leukemias and lymphomas. Moreover, because the present invention improves the antitumor efficacy of 5-FU and 5-FU prodrugs, cancer types that may have shown less than desirable responsiveness to 5-FU previously may show improved responsiveness when administered according to the methods described herein.

It has been unexpectedly discovered that DPD inhibitors such as eniluracil can inhibit the metabolic activation of 5-FU and 5-FU prodrugs and thereby compromise their antitumor activity. Therefore, according to a general aspect of the present invention, by ensuring that 5-FU or 5-FU prodrug levels are in sufficient excess of DPD inhibitor levels at the time the 5-FU or 5-FU prodrug is administered to a patient, the extent to which the DPD inhibitor may interfere with the metabolic activation of the 5-FU or 5-FU prodrug is advantageously minimized, and antitumor efficacy of these agents is thereby improved. Accordingly, in certain embodiments, an irreversible DPD inhibitor such as eniluracil should be dosed at the lowest dose that effectively inactivates DPD and sufficient time should preferably lapse to clear excess inhibitor from the blood stream before dosing with 5-FU. Moreover, as DPD can be substantially eliminated from the patient prior to administration of 5-FU or a 5-FU prodrug, the dose(s) of 5-FU or 5-FU prodrug required to achieve a desired level of therapeutic activity can be significantly reduced, thereby providing economic advantages in addition to the therapeutic advantages described herein.

It will be understood on the part of the skilled artisan, in view of this disclosure, that there exist a multitude of administration and dosing schedules that can be used in the methods described herein while ensuring that the levels of 5-FU or 5-FU prodrug at its time of administration are in a therapeutically effective amount and are in sufficient excess of DPD inhibitor level in the patient to minimize or eliminate inhibition of 5-FU metabolic activation. All such administration and dosing schedules are considered within the scope of the present invention.

In one illustrative embodiment of the invention, a DPD inhibitor is first administered (i.e., pre-dosed) to a patient in need thereof in order to substantially eliminate DPD activity in the patient, followed by administration of 5-FU or a 5-FU prodrug. By “substantially eliminate”, it is meant that the level of DPD activity in the patient is reduced to at least less than 20%, less than 10%, less than 5%, less than 3% or less than 1% relative to the baseline level of DPD activity in the patient prior to administration of the DPD inhibitor. A baseline level of DPD activity for a patient can be readily determined, for example in PBMCs from the patient, using known techniques (e.g., Baker et al., J Clin Oncol 18: 915-926 2000; Schilsky et al., J Clin Oncol 4:1450-7, 1998).

After first administering at least one DPD inhibitor, and thereby substantially eliminating DPD in the patient, 5-FU or a 5-FU prodrug, or a combination thereof, is then administered to the patient after sufficient time has passed to allow the DPD inhibitor to be substantially cleared from the patient by elimination. The delay in time between administration of the DPD inhibitor and the 5-FU or 5-FU prodrug can vary provided that upon administration of the 5-FU or 5-FU prodrug, it is present in the patient in substantial excess relative to the level of DPD inhibitor remaining in the patient at that time. In one illustrative embodiment, the 5-FU or 5-FU prodrug is administered at a dose such that the level of 5-FU or 5-FU prodrug is present in the patient at least in molar excess of the DPD inhibitor remaining in the patient, for example at least about 2-fold, at least about 3-fold, at least about 5-fold or at least about 100-fold excess relative to the level of DPD inhibitor remaining in the patient at the time the 5-FU or 5-FU prodrug is administered. The skilled artisan will recognize that any of a number of known and available techniques may be used for calculating and/or determining the level of excess of 5-FU or 5-FU prodrug in a patient relative to DPD inhibitor in accordance with the embodiments described herein. Such techniques may include, for example, HPLC, LC-MS, ELISA, and others. As noted above, it is believed that by ensuring that the 5-FU or 5-FU prodrug is present in sufficient excess relative to the level of DPD inhibitor in the patient at the time the 5-FU or 5-FU prodrug is administered, interference by the DPD inhibitor with the metabolic activation of the 5-FU or 5-FU prodrug is thereby minimized, and the efficacy of the 5-FU or 5-FU prodrug is thereby improved.

In another embodiment of the invention, the 5-FU or 5-FU prodrug is administered to the patient only after at least 0.1-4 elimination half-lives, 1-4 elimination half-lives, 2-4 elimination half-lives, or 3-4 elimination half-lives of the DPD inhibitor have passed since the DPD inhibitor was administered. The elimination half-lives for certain DPD inhibitors have been determined and, for those that have not, elimination half-lives can be readily determined using well known and established gas-chromatography/mass-spec and HPLC techniques (referenced in Baker et al., J Clin Oncol 18: 915-926 2000; Schilsky et al., J Clin Oncol 4:1450-7, 1998). The elimination half-life for eniluracil in humans has been reported to be about 3.5 hours (e.g., Baker et al., J Clin Oncol 18: 915-926 2000; Ochoa et al., Ann Oncol 11:1313-22, 2000), however it is possible that the half-life for eniluracil and other DPD inhibitors may be dose-dependent and this dose dependency should be considered when determining an appropriate time delay between the administration of DPD inhibitor and 5-FU or 5-FU prodrug. Accordingly, for certain embodiments of the invention that employ eniluracil as the DPD inhibitor, in order to allow the level of eniluracil to be sufficiently decreased by elimination prior to administration of the 5-FU or 5-FU prodrug, the 5-FU or 5-FU prodrug is administered at least about 0.5 hours, about 2 hours, about 4 hours, about 6 hours, about 8 hours, about 10 hours, about 12 hours, about 14 hours, about 24 hours or about 36 hours after administration of the eniluracil. In certain other embodiments, the 5-FU or 5-FU prodrug is administered about 4-72 hours, 4-36 hours, about 4-24 hours, about 4-14 hours, about 6-14 hours or about 8-14 hours after administration of the eniluracil. Of course, it will be understood that these ranges are illustrative in nature and can be varied as necessary or desired for a particular dosing schedule provided that the presence of eniluracil is minimized or absent when 5-FU is dosed.

The DPD inhibitors used in accordance with the present invention may be either reversible or irreversible inhibitors of the DPD enzyme. Illustrative examples of reversible inhibitors of the DPD enzyme include uracil, CDHP and 3-cyano-2,6-dihydroxypyridine (CNDP). Other illustrative reversible DPD inhibitors include those described in U.S. Pat. No. 5,476,855 and WO 95/012400, the contents of which are incorporated herein by reference in their entireties, for example 5-(phenylselenenyl)uracil (PSU), 5-(phenylthio)uracil (PTU), 5-(phenylselenenyl)barbituric acid and 5-(phenylthio)barbituric acid. It will be understood that certain preferred reversible DPD inhibitors include tight-binding inhibitors that dissociate from DPD more slowly than excess inhibitor is eliminated from the body and/or those which inhibit DPD activity but do not substantially inhibit other enzymes that activate fluorouracil, such as uridine phosphorylase (UPase), orotate phosphoribosyltransferase (OPRTase) and thymidine phosphorylase (TP).

In certain preferred embodiments of the invention, the DPD inhibitor is one that irreversibly inactivates the DPD enzyme. Illustrative DPD inhibitors in this regard include, but are not limited to, DPD inhibitors comprising a 5-substituted uracil compound, or a prodrug thereof, particularly a uracil compound substituted in the 5-position by a halogen atom, a C₂₋₄ alkenyl group (e.g., vinyl) optionally substituted by halogen (e.g., 2-bromovinyl, 1-chlorovinyl or 2-bromo-1-chlorovinyl), a C₂₋₆ alkynyl group optionally substituted by a halogen atom, a cyano group, or a C₁₋₄ alkyl group substituted by halogen (e.g., trifluoromethyl).

In a more particular embodiment of the invention, the DPD inhibitor is selected from the group consisting of eniluracil, 5-propynyluracil, 5-cyanouracil, 5-propynyluracil, 5-bromoethynyluracil, 5-(1-chlorovinyl)uracil, 5-iodouracil, 5-bromovinyluracil, (E)-5-(2-bromovinyl)uracil, 5-hex-1-ynyluracil, 5-vinyluracil, 5-trifluorouracil, 5-bromouracil, and 5-(2-bromo-1-chlorovinyl)uracil, or a prodrug thereof.

In another embodiment, the DPD inhibitor is a prodrug of 5-bromovinyluracil, one illustrative compound being represented by the compound I-β-D-arabinofuranosyl-(E)-5-(2-bromovinyl)uracil (also referred to as BV-araU or sorivudine). Certain illustrative prodrug compounds in this regard are described, for example, in U.S. Pat. No. 4,386,076, the disclosure of which is incorporated herein by reference.

In one preferred embodiment of the invention, the DPD inhibitor is eniluracil or a prodrug of eniluracil, such as 5-ethynyl-2(1H)-pyrimidinone (eniluracil missing the 4-oxygen) (Porter, et al., Biochem. Pharmacol 47: 1165-1171, 1994), a nucleoside or deoxynucleoside derivative of eniluracil, a compound that is converted to eniluracil in vivo, and/or a derivative of a DPD inactivator that is converted to the inactivator in vivo. By way of example, such compounds can include nucleoside derivatives which contain a nucleobase corresponding to the above 5-substituted uracil compounds, for example nucleoside derivatives containing a ribose, 2′-deoxyribose, 2′,3′-dideoxyribose, arabinose or other cleavable sugar portion, which may additionally contain a 2′- or 3′-substituent such as a halogen or a 5′ substituent such as an ester. More particular examples of such nucleoside derivatives include 1-(β-D-arabinofuranosyl)-5-prop-1-ynyluracil and 2′,3′-dideoxy-5-ethynyl-3′-fluorouridine.

Numerous 5-FU prodrugs are known which may also be used in accordance with the present invention. A prodrug of 5-FU is a compound which is metabolized in vivo to 5-fluorouracil and may include, by way of illustration, 5-fluorouridine, 5-fluorocytidine, 5-fluoro-2-deoxyuridine, 5-fluoro-2-deoxycytidine, 5-fluoroarabinosyluracil, and their 5′-esters, including phosphate esters. Other illustrative compounds include 5′-deoxy-4′,5-fluorouridine, 5′-deoxy-5-fluorouridine, 1-(2-tetrahydrofuranyl)-5-fluorouracil, a 1-C₁₋₈ alkylcarbamoyl-5-fluorouracil derivative, 1-(2-tetrahydrofuryl)-5-fluorouracil, Ftorafur (Tegafur, an oral 5-FU prodrug that is widely used in Asian countries), and 5′-deoxy-5-fluoro-N-[(pentyloxy)carbonyl]-cytidine (capecitabine, marketed by Roche Laboratories Inc. as Xeloda®), or a compound that is converted to 5-FU in vivo.

In a particularly preferred embodiment of the present invention, the methods described herein employ eniluracil in combination with 5-FU. As noted above, in order to maximize the antitumor activity of 5-FU, eniluracil is administered at a dose to substantially eliminate DPD activity in the patient while also ensuring that eniluracil is not present in excess when 5-FU is dosed. Because eniluracil is a very potent irreversible inactivator of DPD, it is preferably dosed before 5-FU. Eniluracil rapidly inactivates DPD and then is preferably given time to be substantially cleared from the patient by elimination prior to administering the 5-FU. Accordingly, DPD activity will be substantially eliminated and the levels of eniluracil will be advantageously low when 5-FU is administered to the patient.

It will be understood in view of this disclosure that the methods of the present invention can also comprise administration schedules of whatever duration and dosing characteristics desired, provided the dosing schedule is properly selected so that the 5-FU or 5-FU prodrug is present in excess of the level of eniluracil remaining in the patient at the time the 5-FU or 5-FU prodrug is administered. In one illustrative embodiment employing the particular combination of eniluracil and 5-FU, a method of the present invention comprises a once per day for 5 days (5-day) schedule or a once-per-week (weekly) schedule. For 5-day or weekly schedules, the dose-range of eniluracil will typically be between about 0.8 and 10 mg/m², preferably from about 2.5 to 5 mg/m². Note that even higher doses of eniluracil may be used provided that enough time elapses to clear the excess prior to administering 5-FU.

For convenience, a simple fixed dose of eniluracil is preferred in certain embodiments. As described herein, fixed doses in the range of about 2.5-15 mg will generally be appropriate for most patients. In certain preferred embodiments, the fixed dose is in the range of about 2.5-5 mg. Illustrative fixed doses of eniluracil that will eliminate DPD for longer than 24 hours, and in some cases for longer than 3-5 days, in patients of different sizes, are shown in the following Table.

| Body Surface Area | Dose Eniluracil | mg/m² |
|---|---|---|
| (square meters) | (mg) | (range) |
| 0.5 to 1.0 | 2.5 | 2.5 to 5.0 |
| >1.0 to 2.0 | 5.0 | 2.5 to 5.0 |
| >2.0 to 4.0 | 10.0 | 2.5 to 5.0 |

In one illustrative embodiment, the time delay between eniluracil administration and 5-FU administration is between about 4-72 hours, 4-36 hours, 4-24 hours or 4-14 hours (or at least about 1-4 elimination half-lives for eniluracil). For example, eniluracil may be administered the night before 5-FU or, alternatively, can be administered in the morning followed by 5-FU administration in the evening. Using an illustrative dose for 5-FU of about 20 to 30 mg/m² for these schedules (Levin et al., Invest New Drugs 18:383-90, 2000; Schilsky et al., J Clin Oncol 4:1450-7, 1998; Guo et al., Cancer Chemother Pharmacol 52:79-85, 2003), for example, 5-FU should always be in substantial excess relative to eniluracil.

In another embodiment, an extended administration schedule, e.g., a 28-day administration schedule, may be used. One illustrative 28-day b.i.d. schedule (e.g., twice daily for 28 days) involves dosing 5-FU at about 0.5-2 mg/m², preferably about 1 mg/m² (e.g., Baker et al., J Clin Oncol 18: 915-926 2000). To ensure that eniluracil is not present in excess of a low 5-FU dose, eniluracil can be administered at about 2.5 to 5 mg/m² at least about 12 hours before starting the b.i.d. schedule, for example on the evening prior to the first day that 5-FU is administered. Then single daily eniluracil doses of between about 0.5-2 mg/m², for example 1 mg/m², can be administered about 2-8 hours, preferably about 4-6 hours before the first 5-FU dose of each day, for 28 days. This strategy ensures that 5-FU levels from the first dose are in sufficient excess of eniluracil, and that the 5-FU levels from the second 5-FU dose of the day are in sufficient excess of eniluracil. During subsequent treatment days, e.g., days 2 through 28, the level of eniluracil (e.g., when dosed at 1 mg/m²) in the patient's blood should have decreased by about 50% (to 0.5 mg/m²) within four hours (first daily 5-FU dose) and by about 94% (to 0.06 mg/m²) by the second 5-FU dose of the day. The initial 2.5 to 5 mg/m² of eniluracil and the subsequent 1 mg/m² doses should serve to effectively eliminate DPD activity in the patient for the 28 days of 5-FU dosing.

In another illustrative embodiment, eniluracil (or another DPD inhibitor) is first administered and then multiple doses of 5-FU or 5-FU prodrug are thereafter administered at desired time points, before eniluracil is optionally administered again. For example, in an illustrative embodiment, eniluracil is first dosed and then multiple 5-FU doses are administered at illustrative time points of about 12 hours, 36 hours, and even 54 hours thereafter, if desired, before eniluracil is optionally again administered and the cycle repeated.

In a particular embodiment, eniluracil and 5-FU are dosed at a ratio of about 1:5 to 1:15 or 1:8 to 1:12.

In another embodiment of the present invention, 5-FU is dosed relatively soon after eniluracil is administered, e.g., 0-0.5-1 hour, provided in accordance with the present invention that the 5-FU in molar excess relative to eniluracil when administered.

The doses and schedules of eniluracil administration described for embodiments of the present invention differ from those used in the prior Phase II and Phase III human trials (Levin et al., Invest New Drugs 18:383-90, 2000; Schilsky et al., J Clin Oncol: 20:1519-26, 2002). Those studies used higher doses of eniluracil and dosed 5-FU either simultaneously with eniluracil or only one hour after eniluracil. Although some antitumor activity was demonstrated in the Phase III trials, the overall results were less than desired and the trials were not considered successful. It is believed that by using the methods of the present invention, improved efficacy will be achieved by ensuring that excess eniluracil is not present, and thus cannot inhibit 5-FU activation, when the 5-FU or 5-FU prodrug is administered.

The present invention includes as a further feature pharmaceutical formulations comprising at least one pharmaceutically acceptable carrier or excipient and further comprising a DPD inhibitor and/or 5-FU or a 5-FU prodrug, together in a single formulation or present as separate formulations to be administered at separate time points in accordance with the present invention. In one embodiment, the eniluracil and 5-FU are present in one or more formulations at a ratio of about 1:3 to 1:20, 1:5 to 1:15, or 1:8 to 1:12.

A carrier or excipient is “pharmaceutically acceptable” in the sense of being compatible with the other ingredients of the formulation and not injurious to the patient. Formulations include, for example, those adapted for oral, rectal, nasal, topical (including buccal and sublingual), vaginal and parenteral (including subcutaneous, intramuscular, intravenous and intradermal) administration. The formulations may conveniently be presented in unit dosage form and may be prepared by any methods well known in the pharmaceutical arts. Such methods include the step of bringing into association the active ingredient with the carrier that constitutes one or more accessory ingredients. In general, the formulations are prepared by uniformly and intimately bringing into association the active ingredient with liquid carriers or finely divided solid carriers or both, and then if necessary shaping the product.

Formulations according to the present invention may be prepared and/or administered using essentially any available technique. Formulations of the present invention adapted for oral administration, for example, may be presented as discrete units such as capsules, cachets or tablets each containing a predetermined amount of an active ingredient; as a powder or granules; as a solution or a suspension in an aqueous or non-aqueous liquid; or as an oil-in-water liquid emulsion or a water-in-oil liquid emulsion. An active ingredient may also be presented as a bolus, electuary or paste. Oral administration will typically be a preferred route of administration.

A tablet may be made, for example, by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethylcellulose), lubricant, inert diluent, preservative, disintegrant (e.g., sodium starch glycollate, cross-linked povidone, cross-linked sodium carboxymethylcellulose) surface-active or dispersing agent. Molded tablets may be made by molding in a suitable machine a mixture of the powdered compound moistened with an inert liquid diluent. The tablets may optionally be coated or scored and may be formulated so as to provide controlled release of the active ingredient therein using, for example, hydroxypropylmethylcellulose in varying proportions to provide the desired release profile.

Formulations for topical administration in the mouth, for example, include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier. Formulations for rectal administration, for example, may be presented as a suppository with a suitable base comprising for example cocoa butter or a salicylate. Formulation for vaginal administration, for example, may be presented as pessaries, tampons, creams, gels, pastes, foams or spray formulations containing in addition to the active ingredient such carriers as are known in the art to be appropriate.

Formulations for parenteral administration, for example, include aqueous and non-aqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Typically, liquid formulations including one or more active agents are preferably buffered to a pH of 7 to 11, generally 9.5 to 10.5. Certain unit dosage formulations may include those containing a daily dose or unit, daily sub-dose, as hereinabove recited, or an appropriate fraction thereof, of an active ingredient.

Methods for making DPD inhibitors and 5-FU prodrugs described herein are known and may be carried out using conventional methodology. For example, DPD inhibitors referred to above may be prepared by the methods described in Heterocycl. Chem. 19(3) 463-4 (1982) for the preparation of 5-ethynyluracil; J. Chem. Soc. Perkin Trans. 1(16), 1665-70 (1981) for the preparation of 5-(2-bromovinyl)uracil, 5-bromoethynyluracil and 5-(2-bromo-1-chlorovinyl)uracil; Nucleic Acid Chemistry, Vol. 2, 927-30 (1978) for the preparation 5-cyano-uracil; Nucleic Acids Research, 1 (1) 105-7 (1974) for the preparation of 5-vinyluracil; Z. Chern 17(11) 415-16 (1977) for the preparation of 5-trifluoromethyluracil; Nucleic Acids Research 3 (10), 2845 (1976) for the preparation of 5-(1-chlorovinyl)uracil. Certain other compounds of the invention can be prepared in accordance with processes described in European Patent Specification No. 356166 for the preparation of 3′-fluoro-2′,3′-dideoxy-5-alkynyluridine compounds, such as 2′,3′-dideoxy-5-ethynyl-3′-fluorouridine, and European Patent Specification No. 272065 for the preparation of 5-alkynyluracil arabinosides, such as 1-(b-D-arabinofuranosyl)-5-prop-1-ynyluracil. These and other synthetic techniques are known and available for making compounds for use in the present invention.

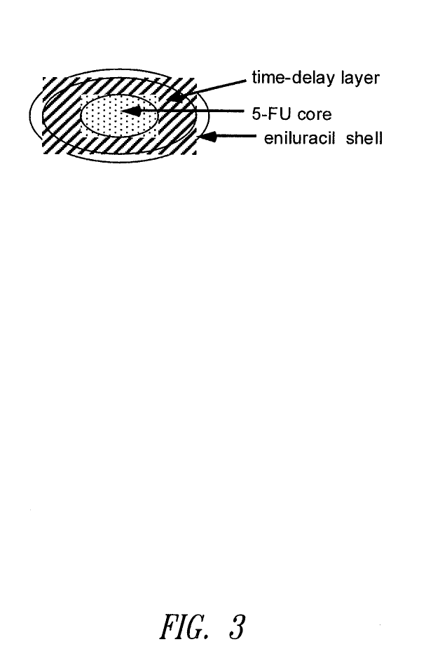

In one embodiment, the present invention provides a combination oral formulation in which a DPD inhibitor and 5-FU or a 5-FU prodrug are dosed together in a manner that allows for the desired temporal release of the components of the formulation into the patient. Differential time-release delivery of two components can be achieved using known techniques and materials. For example, in one embodiment, an oral formulation, e.g., in the form of a tablet, may be composed of three distinct layers, as depicted illustratively in FIG. 3. The outer layer can contain eniluracil in an immediate release formulation. The middle layer can be a time-release component (e.g., time-release buffer) that delays the release of 5-FU or 5-FU prodrug to a desired extent according to the present invention, which 5-FU or 5-FU prodrug is located in the core layer in an immediate release formulation. The DPD inhibitor and 5-FU or 5-FU prodrug are formulated in the proper doses and ratios described herein. In one preferred embodiment, the DPD inhibitor is eniluracil and the 5-FU or 5-FU prodrug is 5-FU or capecitabine.

In another embodiment, an alternative formulation can comprise known delivery vehicles, such as microspheres comprising 5-FU or 5-FU prodrug. In one embodiment, for example, 5-FU or 5-FU prodrug may be encapsulated within a shell of time-release component (e.g., time-release disintegrating buffer) and an outer layer providing immediate release of a DPD inhibitor. In one preferred embodiment, the DPD inhibitor is eniluracil and the 5-FU or 5-FU prodrug is 5-FU or capecitabine. These and other examples of illustrative combination formulations can be designed and made using known techniques to allow the appropriate time-delay between the delivery of the DPD inhibitor and the 5-FU or 5-FU prodrug in a single oral preparation.

In another embodiment, DPD inhibitor may be selectively or preferentially targeted to cancer cells and tumors. The selective effect of DPD in cancer cells and tumors will enable a longer half-life of the compounds relative to their circulating half-life, which will enable a higher steady-state level of activated nucleotides and improved therapeutic index. In one embodiment, selectively targeting DPD inhibitors such as eniluracil to tumors can be accomplished using targeted microspheres, which are well known in the art. In another embodiment, targeting is achieved using a tissue specific receptor such as the asiolglycoprotein receptor to target the compound to hepatocytes and hepatoma cells. Such targeting can be accomplished, for example, by chemically or otherwise modifying the DPD inhibitor (e.g., by placing 3-6 galactose residues on eniluracil).

In another embodiment, the methods described herein further comprise the administration of leucovorin. Leucovorin, or isovorin, the active isomer of leucovorin, is commonly used in conjunction with 5-FU for treating cancer patients. It may also be added to the above-described dosing regimens for eniluracil and 5-FU. Leucovorin has been shown to improve the antitumor efficacy of eniluracil and 5-FU in tumor-bearing rats and in tissue culture (Cao et al., Cancer Res 90:1507-1510, 1993; Fischel et al., Biochem Pharmacol 53: 1703-1709, 1997) and has been administered to patients receiving eniluracil and 5-FU (Schilsky et al., J Clin Oncol 4:1450-7, 1998; Guo et al., Cancer Chemother Pharmacol 52:79-85, 2003). Leucovorin is also advantageously available in an oral formulation.

As noted above, formulations of the present invention include formulations comprising DPD inhibitors that are adapted for topical application. For example, topical formulations may be adapted for delivery to the skin.

In certain embodiments, a topical formulation comprising a DPD inhibitor may be used for reducing the frequency and/or severity of foot syndrome (HFS). The cause of HFS following treatment with 5-FU and 5-FU prodrugs treatment appears to result from catabolic products of 5-FU, most likely FBAL, which are produced by the metabolism of 5-FU in skin tissues. However, some data also suggest that capecitabine induced HFS results from excess activation of capecitabine in keratinocytes via thymidine phosphorylase (Fischel 2004).

Certain DPD inhibitors, such as eniluracil, have been demonstrated to be able to competitively inhibit the anabolic enzymes of uridine phosphorylase (UP) and thymidine phosphorylase (TP), in addition to inhibiting DPD activity. The inhibition of one or more of these enzymes using a topical formulation of the invention could thus interrupt the production of the products needed for causation of HFS.

Thus, in another aspect, the present invention employs topical formulations comprising DPD, TP and/or UP inhibitors, which, when applied using proper dosing and exposure times, are capable of blocking DPD activity in the skin of animals. In certain embodiments, DPD, TP and/or UP activity is locally inhibited in the skin without significantly effecting systemic enzyme activity or 5-FU pharmacokinetics. In other embodiments, DPD, TP and/or UP activity is locally inhibited in the skin and, in addition, systemic enzyme activity is also inhibited to a desired extent. In both scenarios, the topical formulations and methods of the invention may be used in the treatment of hand-foot syndrome (HFS) by blocking the catabolism and anabolism of 5-FU and 5-FU prodrug locally in the skin (e.g., the hands and/or feet) of a patient being treated.

Therefore, the methods of the invention can comprise any application of a topical formulation comprising one or more DPD, TP and/or UP inhibitors that results in a measurable clinical benefit to a patient undergoing treatment with 5-FU or a 5-FU prodrug, generally in the form of preventing the development of HFS, or in reducing the frequency and/or severity of HFS.

In certain embodiments, a DPD inhibitor used in the topical formulations and methods of the present invention is an irreversible inhibitor of the DPD enzyme, such as eniluracil. In some cases, the inhibitor of the DPD enzyme may be a reversible inhibitor of DPD such as CDHP. In certain other embodiments, the DPD inhibitor is an inhibitor of the DPD enzyme and is also an inhibitor of the TP and/or UP enzymes. In other embodiments, a TP and/or UP inhibitor is used in a topical formulation, alone or in combination with a separate DPD inhibitor.

The specific dose of DPD, TP and/or UP inhibitor(s) present in a topical formulation of the invention, and optimal exposure time, may, of course, vary depending upon the particular inhibitor(s) used as well as the particular components employed in the formulation, however these are readily determinable by a skilled artisan in view of the present disclosure. If a topical formulation is effective for the local inhibition of DPD and/or other enzyme activity in the skin of an animal and is also effective for achieving a desired level of systemic DPD inhibition (if such inhibition is desired), then such formulation is considered within the spirit and scope of the present invention.

In certain embodiments, an effective dose of a DPD, TP and/or UP inhibitor, in the topical formulations of the invention, is a dose effective for inhibiting DPD, TP and/or UP activity in the skin (e.g., hands and/or feet) of a patient without effecting substantial systemic inhibition (e.g., systemic DPD inhibition is less than about 40%, 10%, 5% or 1% of the normal DPD activity in the patient). Of course, an effective dose will also preferably be one that prevents the development of HFS, reduces the frequency of HFS and/or reduces the severity of HFS.

In certain embodiments, doses of inhibitors present in a topical formulation of the invention may be from about 0.0001 to about 0.5 w/w. In a more particular embodiment, the dose in the topical formulation is from about 0.0001 to about 0.05 w/w. In a more particular embodiment, the dose in the topical formulation is from about 0.0001 to about 0.01 w/w. In a more particular embodiment, the dose in the topical formulation is from about 0.001 to about 0.05 w/w. In another more particular embodiment, the dose in the topical formulation is from about 0.001 to about 0.01 w/w.

The topical formulation may be applied bilaterally to hands (e.g., wrist down) and feet (e.g., ankles down). In certain embodiments, it will be desired to concentrate the majority of the topical formulation on the palms of the hands and the soles of the feet. The topical formulation may be massaged into or otherwise contacted with the skin for any suitable duration provided the exposure time is not such that systemic DPD activity is substantially inhibited. In certain embodiments, the duration of exposure time to the topical formulation is about 1-10 minutes, 1-5 minutes or 1-3 minutes per appendage, followed by removal of any excess ointment off the hand and foot.

In certain embodiments, the topical formulation may be applied one or multiple times provided that the application allows for effective inhibition of skin DPD, TP and/or UP activity but does not result in substantial systemic inhibition. For example, the topical formulation may be administered once, twice, three times, four times, five times or more, as needed or desired, during each course of 5-FU or 5-FU prodrug treatment.

The topical formulations of the invention may comprise essentially any suitable components that are biologically compatible and that are effective for facilitating the local delivery of the DPD, TP and/or UP inhibitor(s) in the skin, particularly the hands and feet, of a patient. Thus, the topical formulations may be in any convenient format, including ointments, creams, lotions, aerosol sprays, roll-on liquids, sticks, pad forms, etc., as long as local delivery of the inhibitor is achieved as described herein. Topical formulations may further comprise expedients which inhibit the systemic absorption of the inhibitor(s) from the topical site(s). Examples include, for example, vasoconstrictors such as epinephrine which reduce or delay absorption of the inhibitor.

In certain embodiments, the topical formulations may be anhydrous or emulsions, such as oil and water emulsions. Whether anhydrous or emulsion type, formulations may further include any of a variety of pharmaceutically acceptable carriers, skin actives and/or other necessary or desired components.

Generally, suitable amounts of a given carrier may range, for example, from about 1 to about 99%, from about 5 to about 70%, from about 10 to about 40% by weight, etc. Illustrative carriers may include, but are not limited to, emollients, water, inorganic powders, foaming agents, emulsifiers, fatty alcohols, fatty acids, and the like, as well as combinations thereof.

Emollients include substances selected from, for example, polyols, esters and hydrocarbons.

Illustrative polyols include, for example, propylene glycol, dipropylene glycol, polypropylene glycol, polyethylene glycol, sorbitol, hydroxypropyl sorbitol, hexylene glycol, 1,3-butylene glycol, 1,2,6-hexanetriol, glycerin, ethoxylated glycerin, propoxylated glycerin, xylitol, and the like, as well as mixtures thereof.

Illustrative esters useful as emollients include, for example:

(1) Alkyl esters of fatty acids having about 10 to 20 carbon atoms. For example, methyl, isopropyl, and butyl esters of fatty acids may be used. Particular examples include hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, cetyl lactate, and the like. In certain embodiments, C12-C15 alcohol benzoate esters are used.

(2) Alkenyl esters of fatty acids having about 10 to 20 carbon atoms, illustrative examples including oleyl myristate, oleyl stearate and oleyl oleate.

(3) Ether-esters such as fatty acids esters of ethoxylated fatty alcohols.

(4) Polyhydric alcohol esters, such as ethylene glycol mono and di-fatty acid esters, diethylene glycol mono- and di-fatty acid esters, polyethylene glycol (200-6000) mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

(5) Wax esters such as beeswax, spermaceti, myristyl myristate, stearyl stearate.

(6) Sterol esters, of which cholesterol fatty acid esters are examples thereof.

Illustrative hydrocarbons include, for example, mineral oil, polyalphaolefins, petrolatum, isoparaffin, polybutenes, and the like, as well as mixtures thereof.

Inorganic powders may also be used as carriers, alone or in conjunction with other carriers, examples of which include clays (such as Montmorillonite, Hectorite, Laponite and Bentonite), talc, mica, silica, alumina, zeolites, sodium sulfate, sodium bicarbonate, sodium carbonate, calcium sulphate, etc., and mixtures thereof.

Aerosol propellants may also be used as carriers. Propellants are normally based on volatile hydrocarbons such as propane, butane, isobutene, pentane, isopropane and mixtures thereof. Philipps Petroleum Company is a source of such propellants under trademarks including A31, A32, A51 and A70. Halocarbons including fluorocarbons and dimethyl ether represent other illustrative propellants.

Emulsifiers may constitute at least a portion of the carrier for compositions according to the present invention, illustrative examples of which include, nonionic, anionic, cationic, or amphoteric emulsifying agents. They will typically range in amounts anywhere from about 0.1 to about 20% by weight, however this may vary depending on the particular emulsifier and the context of its intended use. Illustrative nonionic emulsifiers include, for example, alkoxylated compounds based on C10-C22 fatty alcohols and acids and sorbitan. These materials are available, for instance, from the Shell Chemical Company under the Neodol trademark. Copolymers of polyoxypropylenepolyoxyethylene sold by the BASF Corporation under the Pluronic trademark are sometimes also useful. Alkyl polyglycosides available from the Henkel Corporation represent additional illustrative emulsifiers.

Illustrative anionic type emulsifiers include, for example, fatty acid soaps, sodium lauryl sulphate, sodium lauryl ether sulphate, alkyl benzene sulphonate, mono- and di-alkyl acid phosphates, sarcosinates, taurates and sodium fatty acyl isethionate.

Illustrative amphoteric emulsifiers include, for example, such materials as dialkylamine oxide and various types of betaines (such as cocamidopropyl betaine).

Preservatives such as methyl paraben and propyl paraben may also be used, as desired, for example to prevent microbial contamination.

In a specific embodiment of the invention, the topical formulation is a petroleum-based eniluracil ointment compounded, for example, using a commercial base (e.g., Aquaphor®: petrolatum, mineral oil, cresin, and lanolin alcohol).

The invention can be further understood upon consideration of the following non-limiting Example.

### EXAMPLES

### Example 1

### Excess Eniluracil Diminishes the Antitumor Activity of Eniluracil and 5-FU

Rats were implanted with Ward carcinoma tumors and were treated with one of the following regimens after their tumors grew to 3,000 mg in weight as previously described (Cao et al., Cancer Res 90:1507-1510, 1993). Rats bearing 3,000 mg of tumor mass were dosed at Day 0, Day 7, and Day 14 with the following treatments.

| | TREATMENT | | |
|---|---|---|---|
| | | Eniluracil at | 5-FU at |
| STUDY ARM | Eniluracil | 55 minutes | 60 minutes |
| Group | (mg/kg) | (mg/kg) | (mg/kg) |
| A | 0 | 0 | 0 |
| B | 1 | 0 | 5 |
| C | 1 | 25 | 5 |

Group A rats received no treatment. Group B rats were intraperitoneally (ip) dosed with 1 mg/kg eniluracil (time (t)=0) followed by intravenous (iv) 5 mg/kg 5-FU at t=60. Group C rats were dosed ip with 1 mg/kg eniluracil (t=0) followed by 25 mg/kg eniluracil ip at t=55 minutes and by 5 mg/kg 5-FU iv at t=60 minutes. Animals were dosed once per week for three weeks. Eniluracil was also dosed ip at 1 mg/kg to rats in Groups B & C on Days 2 and 3 of each weekly treatment. The treatment for Group C mimicked the clinical trials where high levels of eniluracil were present when 5-FU was dosed.

The tumors in Group A rapidly grew to 10,000 mg and the rats were sacrificed. Tumors in Group B were rapidly eliminated while those in Group C on average decreased only slightly in size. These results demonstrate that the excess eniluracil in Group C greatly diminished the antitumor activity 5-FU, likely due to interference of eniluracil with the metabolic activation of 5-FU.

### Example 2

### Eniluracil Inhibits the Metabolic Activation of 5-FU to Active Nucleotides

HEK 293 cells were initially treated with eniluracil (10 μM) for 1 hour. After a washout period ranging from 4-48 hours, cells were treated with [6-¹⁴C]-5-FU (66 μM) for 2 hours at 37 degrees C. Controls were HEK 293 cells treated for 2 hours either with [6-¹⁴C]-5-FU (66 μM) alone, or with eniluracil (10 μM) and [6-¹⁴C]-5-FU (66 μM) co-administered together without preincubation. Reverse phase HPLC with radioactivity detection was utilized to quantify [6-¹⁴C]-5-FU catabolites/anabolites present in cell lysates. In a separate set of experiments cytotoxicity of 5-FU at different eniluracil dose schedules was examined. HEK 293 cells were treated with a range of 5-FU concentrations for 72 hours at 37 degrees C. following 1 hour eniluracil (5 μM) preincubation, without eniluracil, or with eniluracil (5 μM) co-administered without preincubation. Cytotoxicity was assessed by the MTS proliferation assay and the EC₅₀ values for 5-FU calculated.

Pre-treatment of cells with eniluracil for 1 hour, followed by a 36 hour or a 48 hour wash out prior to [6-¹⁴C]-5-FU treatment produced significant increases in [6-¹⁴C]-FUMP anabolite formation compared to [6-¹⁴C]-5-FU treatment alone, or eniluracil and [6-¹⁴C]-5-FU co-administration (p-values<0.05). For example, the 48 hour washout produced a 70% increase in [6-¹⁴C]-FUMP formation versus [6-¹⁴C]-5-FU treatment alone, and a 41% increase in [6-¹⁴C]-FUMP formation versus eniluracil and [6-¹⁴C]-5-FU co-administration. In cytotoxicity experiments, pre-treatment with eniluracil for 1 hour increased the cytotoxicity of 5-FU with a decreased EC₅₀ value (264.0 nM±1.7) (mean ±SE) compared to the 5-FU treatment alone (311.4 nM±1.1) or the EU and 5-FU co-administration without preincubation (307.8 nM±1.03) (p<0.05).

These results further confirm a role for eniluracil in inhibiting the anabolic pathways that convert 5-FU to active anti-tumor nucleotides, in addition to its known role in inhibiting the catabolic pathways that degrade 5-FU to inactive forms.

### Example 3

### Eniluracil Causes Plasma Uridine Levels to Increase

While eniluracil inhibition is known to cause an increase in uracil levels due to DPD inhibition, inhibitory effects on other enzymes that anabolize fluoropyrimidines would be expected to quantitatively alter the levels of other anabolites, such as uridine. Eniluracil was administered to mice at 2 mg/kg, 25 mg/kg and 100 mg/kg. Plasma samples were taken at 0 minutes, 15 minutes, 30 minutes, 60 minutes, 2 hours, 24 hours and 48 hours. Uridine levels were determined by LC-MS technology using known techniques and standards were used to validate the assay.

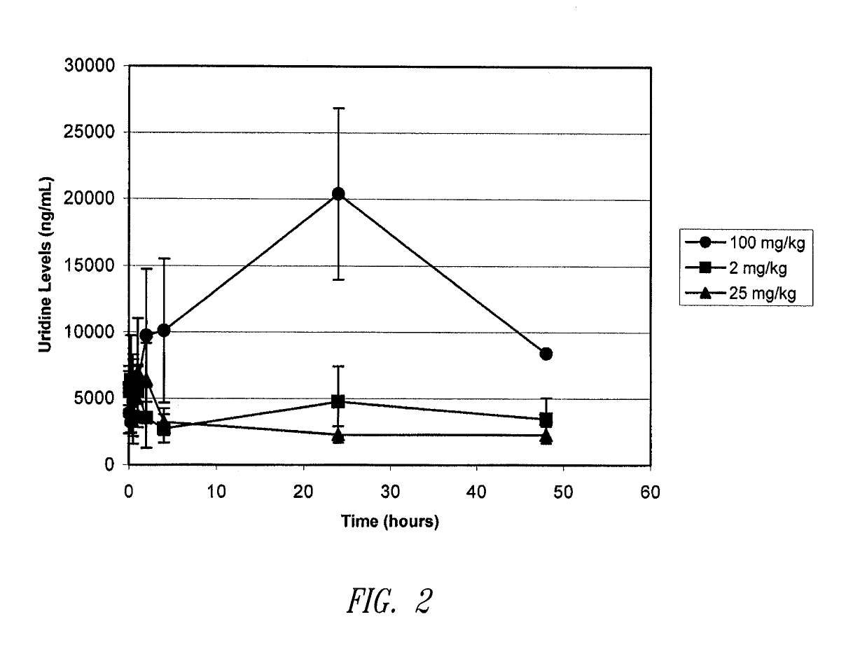

Results from these experiments, shown in FIG. 2, demonstrate that eniluracil causes an increase in uridine levels following administration. This finding is consistent with eniluracil having an inhibitory effect on anabolic enzymes such as uridine phosphorylase, and further supports a role for eniluracil in inhibiting the anabolic conversion of 5-FU to active nucleotides.

### Example 4

### In-Vitro Determination of Eniluracil Dermal Irritation

The EpiDerm™ skin model system (MatTek) was used to assess the potential dermal irritation of eniluracil alone or its formulation. This system consists of normal, human-derived epidermal keratinocytes (NHEK) which have been cultured to form a multilayered, highly differentiated model of the human epidermis. The model contains organized basal, spinous, granular, and cornified layers analogous to those found in vivo, and exhibits in vivo-like morphological and growth characteristics which are uniform and highly reproducible.

The EpiDerm™ system is mitotically and metabolically active. Markers of mature epidermis-specific differentiation such as pro-filaggrin, the K1/K10 cytokeratin pair, involucrin, and type I epidermal transglutaminase have been localized in the model. The MTT (3-[4,5-dimethylthiazol-2-yl]-2,5-diphenyltetrazolium bromide) conversion assay, which measures the NAD(P)H-dependent microsomal enzyme reduction of MTT (and to a lesser extent, the succinate dehydrogenase reduction of MTT) to a blue formazan precipitate, was used to assess cellular metabolism after exposure to a test article for various exposure times. The duration of exposure resulting in a 50% decrease in MTT conversion in test article-treated EpiDerm™ cultures, relative to control cultures, was determined (ET₅₀).

Eniluracil was prepared and administered to the test system at the three final concentrations of 1000 μM, 500 μM, and 100 μM, and tested at three exposure times of 8, 20, and 24 hours. The DMSO solvent control was tested at exposure times of 8 and 24 hours. The negative control was tested at exposure times of 4 and 24 hours, and the positive control was tested at exposure times of 4 and 8 hours. The results of the trial, presented in Table 1, confirmed that the three doses of eniluracil were well-tolerated by the EpiDerm™ skin model at topical exposures of up to 24 hours. The ET₅₀ value for the positive control, 1% Triton®-X-100, fell within two standard deviations of the historical mean (4.17 to 6.96 hours), thereby meeting the acceptance criteria. Based upon the results of other chemicals tested in this assay system historically, ET₅₀ values of greater than 24 hours are suggestive of materials with low acute dermal irritation potential. Within the confines of this study, the test articles resulted in ET₅₀ values notably longer than that obtained from the positive control, 1% Triton®-X-100.

TABLE 1
ET₅₀ values of various concentrations of eniluracil
tested in the EpiDerm ™ skin model

| Compound | ET₅₀ | |
|---|---|---|
| Concentration | (hours) | pH |
| Eniluracil (1000 uM) | >24 | 5 |
| Eniluracil (500 uM) | >24 | 5 |
| Eniluracil (100 uM) | >24 | 5 |
| 1% Triton-X-100 | 6.09 | NA |

### Example 5

### Evaluation of Eniluracil Ointment in the EpiDerm™ Skin Model

EpiDerm™ cultures were tested in duplicate with eniluracil ointment at four exposure times of 4, 8, 16, and 24 hours. Eniluracil ointment was prepared by dissolving appropriate amounts of eniluracil in a sodium hydroxide solution and then levigating it with Aquaphor®. to obtain 0.0005-0.1% w/w. Hydrochloric acid in an amount equivalent to the sodium hydroxide was added to neutralize the ointment. The exposure time control was also exposed in duplicate for 4 and 24 hours. Table 2 below summarizes the ET₅₀ results of the EpiDerm™ assay for the test articles and the positive control, using the negative control results to determine the relative viability. Additionally, for the test articles, eniluracil 0.1% (w/w) and eniluracil 0.0005% (w/w), percent of control values were calculated using the test article, placebo ointment, as the placebo or vehicle control. The ET₅₀ value for the positive control, 1% Triton®-X-100, fell within two standard deviations of the historical mean (4.17 to 6.96 hours), thereby meeting the acceptance criteria. Finally, none of the test articles were observed to directly reduce MTT in the absence of viable tissue. Test article residues persisted on some of the treated tissues following the rinsing process at all exposure times for the test article, placebo ointment, and at the 8, 16, and 24 hour exposure times for the test articles, eniluracil 0.1% (w/w) and eniluracil 0.0005% (w/w). The test article residues presumably prolonged the exposure times relative to the reported exposure times.

TABLE 2
ET₅₀ values of various concentrations of eniluracil
tested in the EpiDerm ™ skin model

| | ET₅₀ | |
|---|---|---|
| Compound Concentration | (hours) | pH |
| Placebo Ointment | >24 | 5 |
| Eniluracil 0.1% (w/w) | >24 | 5 |
| Eniluracil 0.0005% (w/w) | >24 | 5 |
| 1% Triton-X-100 | 4.95 | NA |

### Example 6

### Evaluation of Topical Eniluracil in Mice

To evaluate the effect of topical administration of eniluracil on DPD activity in the skin and in the liver, various studies were conducted in mice. The DPD activity in the skin of mice treated with placebo was determined to be 1.4 pmol/min/mg (mean of DPD activity of placebo in Table 3) of protein and in the liver it was determined to be 2426.66 pmol/min/mg of protein (mean of DPD activity of placebo in Table 4). Mice in the studies had an exposure time of one hour i.e. eniluracil ointment was applied for one hour and then removed, unless otherwise specified.

DPD activity was measured according to the following procedure. All tissue samples were homogenized in ice-cold buffer (35 mM KH₂PO₄ buffer 1.5 mM DTT, pH=8) in the presence of protease inhibitors, centrifuged for 1 hour at 100,000×g at 4° C., and the supernatant (cytosolic fraction) was collected for use as the enzyme source. The reaction mixture for determining DPD activity in tissue samples consisted of 35 mM KH₂PO₄ buffer (pH=8), 5 mM MgCl₂, 1 mM DTT, 100 μM NADPH, 20 μM [6-¹⁴C]-5-FU, and 80 μL cytosolic extract in a final volume of 160 μL. All incubations were initiated by the addition of cytosolic extract and were conducted at 37° C. in a shaking water bath. Enzymatic activity were terminated after 15 to 30 minutes by boiling ×3 min and subsequent frozen at −80° C. Precipitated protein was removed by centrifugation, the supernatant was filtered and 5-FU catabolites [6-¹⁴C]-5-fluoroureidopropionic acid and [6-¹⁴C]-dihydro-5-fluorouracil formed by DPD, were separate by reverse-phase HPLC (mobile phase: 5 mM tetrabutylammonium hydrogensulfate and 1.5 mM potassium phosphate [pH=7.6]), and quantified by a Flow Scintillation Analyzer connected on line with the HPLC.

### A. Determination of Dose Range for Topical Eniluracil:

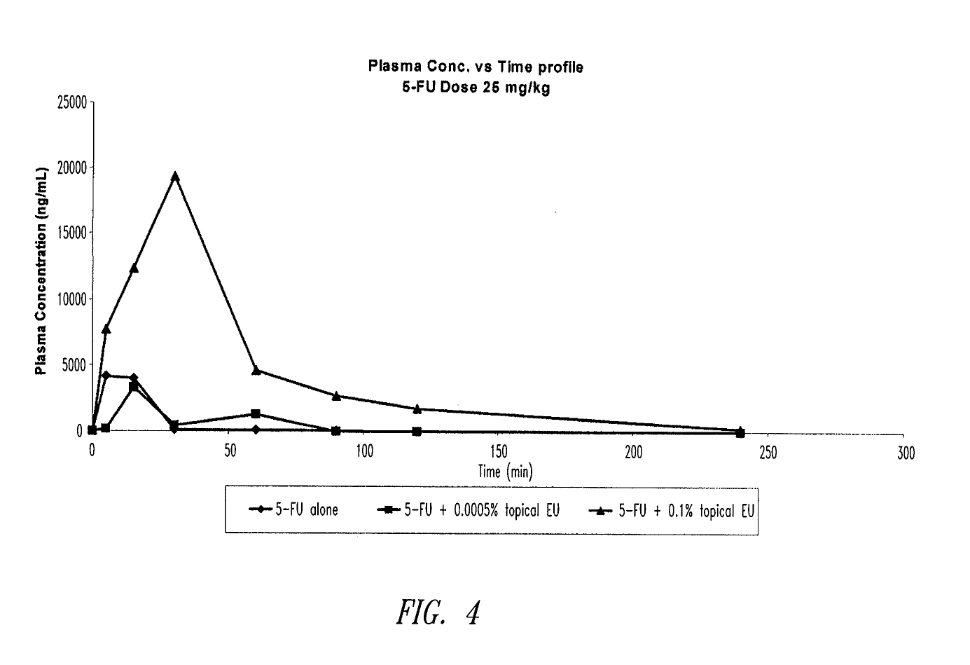

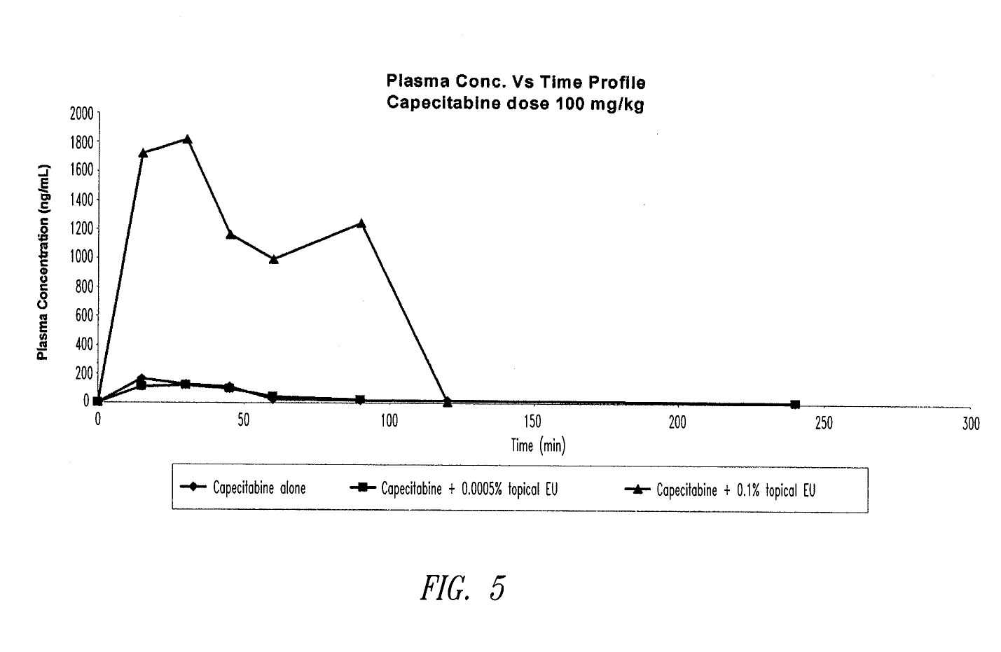

The dose range for topical administration of eniluracil in mice was determined by dosing 40-50 mg of placebo, 0.0005% w/w and 0.1% w/w of eniluracil ointment for one hour and then removing it with alcohol swab. Skin and liver samples were then collected at pre-determined time, and DPD activity was measured. Effects on skin DPD activity and liver DPD activity are listed in Table 3 and 4. These animals were also dosed with 25 mg/kg of 5-FU (administered after one hour of ointment application) to evaluate the effect of 0.1% w/w and 0.0005% w/w on its pharmacokinetics. 5-FU plasma levels were determined. A plasma concentration time profile for 5-FU is shown in FIG. 4. A similar pharmacokinetic evaluation was also conducted with 100 mg/kg capecitabine (administered after one hour of ointment application). A plasma concentration time profile for 5-FU after administration of capecitabine is shown is FIG. 5. It is clear from the data that 0.0005% w/w had no effect on the skin and liver DPD activity, and it did not affect the pharmacokinetics of 5-FU (FIG. 4). On the other hand, 0.1% w/w totally inhibited DPD activity in skin and in liver, and it also affected the 5-FU pharmacokinetics. Similar effects were seen when the animals were dosed with capecitabine i.e. 0.0005% w/w did not affect the 5-FU pharmacokinetics after capecitabine administration but 0.1% w/w did affect the 5-FU pharmacokinetics after capecitabine administration (FIG. 5).

TABLE 3
DPD activity in the skin of the mice treated with (0.0005,
and 0.1% (w/w)) of eniluracil ointment for 1 hour
Skin DPD activity (pmol/min/mg of protein)

| Time | | 0.0005% | 0.1% |
|---|---|---|---|
| Points | Placebo | (w/w) | (w/w) |
| 5 min | 1.74 | 1.41 | ND |
| 5 min | 1.70 | 1.30 | ND |
| 2 hrs | 1.18 | 1.29 | 0.33 |
| 2 hrs | 1.12 | 1.15 | 0 |
| 4 hrs | 1.53 | 1.64 | 0 |
| 4 hrs | 1.41 | 1.54 | 0 |

TABLE 4
DPD activity in the liver of the mice treated with (0.0005,
and 0.1% (w/w)) of eniluracil ointment for 1 hour
Liver DPD activity (pmol/min/mg of protein)

| Time | | 0.0005% | 0.1% |
|---|---|---|---|
| Points | Placebo | (w/w) | (w/w) |
| 5 min | 2339.53 | 2088.71 | 0 |
| 5 min | 2271.07 | 2227.77 | 0 |
| 2 hrs | 2516.14 | 1827.22 | 0 |
| 2 hrs | 2579.91 | 1770.27 | 0 |
| 4 hrs | ND | 1691.36 | 0 |
| 4 hrs | ND | 1598.79 | 92.14 |

### B. Effect of Various Doses of Eniluracil Ointment on Skin and Liver DPD Activity

To evaluate the effect of various doses of topical eniluracil on skin and liver DPD activity, 40-50 mg of eniluracil ointment (0.001, 0.005, 0.01 and 0.05% w/w) was applied topically for one hour and then removed using an alcohol swab. Skin and liver samples were collected and DPD activity was measured. DPD activity was affected in both liver and skin (Table 5 and 6), except 0.001% w/w which affected DPD activity (33% inhibited) in skin only; DPD activity in liver remained unaffected. This demonstrates that in the mice where absorption of drug is rapid, 40-50 mg of 0.001% w/w is sufficient enough to block 33% of skin DPD activity without affecting the liver DPD activity.

TABLE 5
DPD activity and percent of DPD activity inhibited
in the liver of the mice treated with (0.001, 0.005,
0.01 and 0.05% (w/w)) topical eniluracil

| | | DPD Activity | Percent of DPD |
|---|---|---|---|
| Group | | (pmol/min/mg protein) | activity inhibited |
| 0.001% | w/w | 3009.14 | −24.00 |
| 0.001% | w/w | 2890.07 | −19.10 |
| 0.005% | w/w | 1625.52 | 33.01 |
| 0.005% | w/w | 1550.92 | 36.09 |
| 0.01% | w/w | 159.89 | 93.41 |
| 0.01% | w/w | 133.19 | 94.51 |
| 0.05% | w/w | 0 | 100.00 |
| 0.05% | w/w | 0 | 100.00 |

TABLE 6
DPD activity and percent of DPD activity inhibited
in the skin of the mice treated with (0.001, 0.005,
0.01 and 0.05% (w/w)) topical eniluracil

| | | DPD Activity | Percent of DPD |
|---|---|---|---|
| Group | | (pmol/min/mg protein) | activity inhibited |
| 0.001% | w/w | 0.95 | 32.07 |
| 0.001% | w/w | 0.91 | 34.79 |
| 0.005% | w/w | 1.14 | 18.29 |
| 0.005% | w/w | 0.89 | 36.07 |
| 0.01% | w/w | 0.71 | 49.07 |
| 0.01% | w/w | 0 | 100.00 |
| 0.05% | w/w | 0 | 100.00 |
| 0.05% | w/w | 0 | 100.00 |

C. Recovery of DPD Activity after 0.01% w/w Eniluracil Ointment:

To evaluate the recovery of DPD in the skin and in the liver, mice were treated with 0.01% w/w of eniluracil ointment (this dose was high enough to block significant amount of DPD activity in the skin and in the liver) for one hour and then the ointment was removed using an alcohol swab Skin and liver samples were collected and were analyzed for DPD activity. The results in Table 7 show that after 1 hour of application of 0.01% w/w of eniluracil ointment DPD activity was totally inhibited in skin and DPD activity in the liver was also significantly affected (Table 8). DPD activity in both the tissues returned to normal activity after day 6 for liver and day 4 for skin.

TABLE 7
DPD activity in the skin of the mice treated
with 0.01% w/w topical eniluracil

| | Mean DPD Activity | Percent of DPD |
|---|---|---|
| Time Points | (pmol/min/mg protein) | activity inhibited |
| Day 0 (1 hr after | 0.00 | 100.00 |
| application) | | |
| Day 2 | 1.34 | 4.29 |
| Day 4 | 1.62 | −15.71 |
| Day 6 | 2.75 | −96.43 |
| Day 8 | 3.04 | −117.14 |

TABLE 8
DPD activity in the liver of the mice treated
with 0.01% w/w topical eniluracil

| | Mean DPD Activity | Percent of DPD |
|---|---|---|
| Time Points | (pmol/min/mg protein) | activity inhibited |
| Day 0 (1 hr after | 41.72 | 98.28 |
| application) | | |
| Day 2 | 1172.48 | 51.68 |
| Day 4 | 1157.53 | 52.30 |
| Day 6 | 2920.92 | −20.37 |
| Day 8 | 3175.38 | −30.85 |

D. Effect on Skin and Liver DPD after a Short Exposure of 0.005% w/w Eniluracil Ointment:

In our earlier study, 0.005% w/w of eniluracil ointment was effective to block ˜33% of DPD activity in the skin and 27% in the liver after 1 hour of application. In this study we evaluated the effect on DPD activity in the skin and the liver of mice treated with 0.005% (w/w) topical eniluracil for 2, 5, 15 and 30 minutes. Table 9 shows that DPD activity in the skin was affected even after a short exposure of topical eniluracil. Table 9 also shows that DPD activity in the liver was not affected after short exposure of topical eniluracil. The data is consistent with our previous study and also demonstrates that in mice a longer exposure is not needed to completely inhibit DPD activity in the skin.

TABLE 9
DPD activity and percent of DPD activity inhibited in the
skin and the liver of the mice treated with 0.005% w/w
of topical eniluracil for 2, 5, 15, and 30 minutes.

| | | Skin DPD | | Liver DPD | Percent |
|---|---|---|---|---|---|
| | | activity | | activity | of DPD |
| | | (pmol/min/mg | Percent | (pmol/min/mg | activity |
| Time | | protein) | inhibited | protein) | inhibited |
| 2 | min | 0 | 100.00 | 2657.72 | −9.52 |
| 2 | min | 0 | 100.00 | 2849.44 | −17.42 |
| 5 | min | 1.445 | −3.21 | 3248.27 | −33.86 |
| 5 | min | 1.481 | −5.79 | 3126.83 | −28.85 |
| 15 | min | 0.938 | 33.00 | 3154.15 | −29.98 |
| 15 | min | 0.496 | 64.57 | 3151.17 | −29.86 |
| 30 | min | 0 | 100.00 | 3138.76 | −29.34 |
| 30 | min | 0.78 | 44.29 | 2969.64 | −22.38 |

### Example 7

### Evaluation of Topical Eniluracil in Pigs

To evaluate the effect of single administration of topical eniluracil on skin DPD activity, pigs were dosed with 5 gms of 0.0005, 0.001, 0.005 and 0.01% w/w for 30 minutes and then the ointment was wiped off using a Kimwipe®. Skin samples were collected at predetermined time points. To reduce the intra-pig variability, each pig was dosed with treatment on one site and placebo on the other. Table 10 shows the effect of individual doses on skin DPD activity at 0.5 hrs and 12 hours. (7 hours for the pig treated with 0.001% w/w). DPD activity was measured following the procedure noted above. It is clear from the data that all dose levels of eniluracil affected skin DPD activity and most of the DPD activity was recovered after 12 hours.

TABLE 10
Percent of DPD activity inhibited in the skin of
the pigs treated with 0.0005, 0.001, 0.005 and 0.01%
w/w of topical eniluracil for 30 minutes.

| | | | |
|---|---|---|---|
| 0.0005% | 0.001% | 0.005% | 0.01% |
| w/w | w/w | w/w | w/w |

| | |
|---|---|
| Time Points | Percent of DPD activity inhibited |

| | | | | | |
|---|---|---|---|---|---|
| 0.5 | hrs | 53.55 | 44.01 | 87.24 | 79.42 |
| 0.5 | hrs | 55.90 | 45.38 | 90.84 | 79.57 |
| 12 | hrs | 36.72 | 61.11* | 16.55 | −22.52 |
| 12 | hrs | 34.60 | 61.64* | 21.08 | −21.74 |

*7 hours not 12 hours

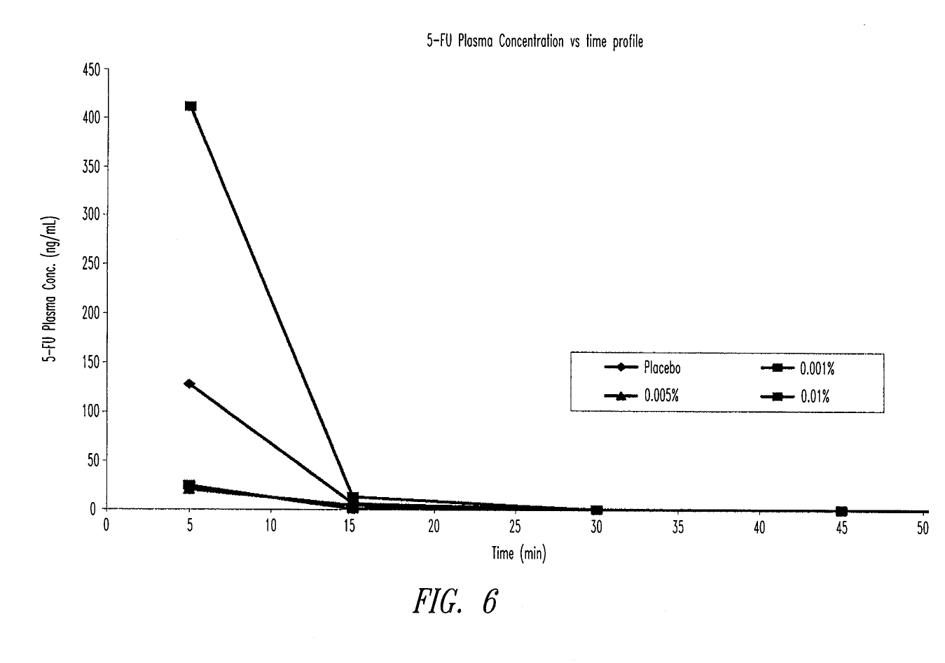

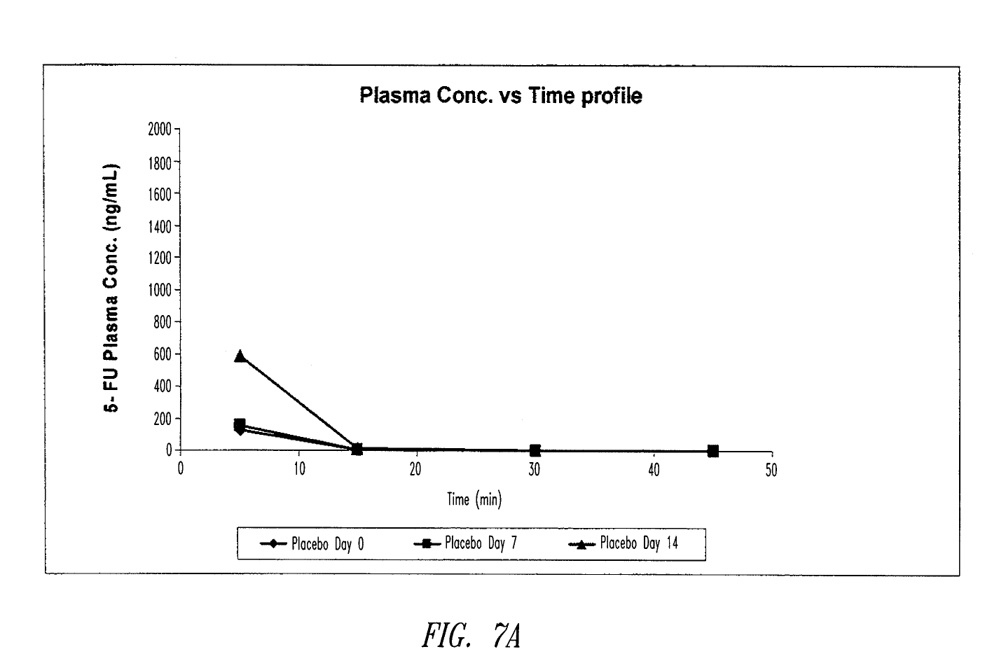

In another experiment, pigs were dosed with 0.001, 0.005 and 0.01% w/w of topical eniluracil for 30 minutes and then the ointment was wiped off using a Kimwipe®. These animals were also dosed with 0.5 mg/kg of 5-FU and blood and PBMC samples were collected at pre-defined time intervals. 5-FU plasma levels and DPD activities were determined. PBMC data in Table 11 shows that DPD activity was quite high in the treated animals compared to the placebo. Also, FIG. 6 shows that a single administration of topical eniluracil had no effect on 5-FU pharmacokinetics.

These data indicate that in pigs where absorption is slower compared to mice, DPD activity in the skin was inhibited whereas the systemic DPD activity remained unchanged. These animals continued to receive placebo, 0.001, 0.005 and 0.01% w/w of topical eniluracil, for 30 minutes and skin DPD activity was measured at Day 7 and 14. These animals were also dosed with 0.5 mg/kg of 5-FU on Day 7 and on Day 14

TABLE 11
DPD activity in the PBMCs of the pigs treated with 0.001,
0.005 and 0.01% w/w of topical eniluracil for 30 minutes.

| | | | 0.001% | 0.005% | 0.01% |
|---|---|---|---|---|---|
| | | Placebo | (w/w) | (w/w) | (w/w) |
| 0 | hrs | 45.26 | 58.29 | 102.22 | ND |
| 0 | hrs | 29.81 | 57.21 | 62.60 | ND |
| 0.5 | hrs | 26.64 | 58.07 | 97.89 | 127.68 |
| 0.5 | hrs | 33.69 | 32.14 | 54.12 | 128.58 |
| 5 | hrs | 73.13 | 50.13 | 67.92 | 63.50 |
| 5 | hrs | 43.24 | 45.52 | 78.51 | 125.39 |

Following repeated administration of topical eniluracil (0.001, 0.005 and 0.01% w/w) for 30 min every day, for 6 days followed by a skin biopsy results, showed that on Day 7, i.e. after 24 hours there was no effect on the skin DPD activity; however, after 14 days of treatment with topical eniluracil (0.01% w/w 30 minutes every day) reduced DPD activity in the skin by approximately 15%. (Table 12).

TABLE 12
DPD activity in the pig skin treated with 0.001, 0.005 and 0.01%
w/w of topical eniluracil for 30 minutes every day for 13 days and
sample was collected on day 14, 24 hours after administration.

| Time | | 0.001% | 0.005% | 0.01% |
|---|---|---|---|---|
| points | Placebo | (w/w) | (w/w) | (w/w) |
| 7 days | 24.893 | 65.59 | 39.24 | 31.66 |
| 7 days | 25.006 | 73.99 | 39.55 | 33.93 |
| 14 days | 23.799 | 53.27 | 58.08 | 8.72 |
| 14 days | 28.695 | 53.40 | 77.01 | 5.83 |

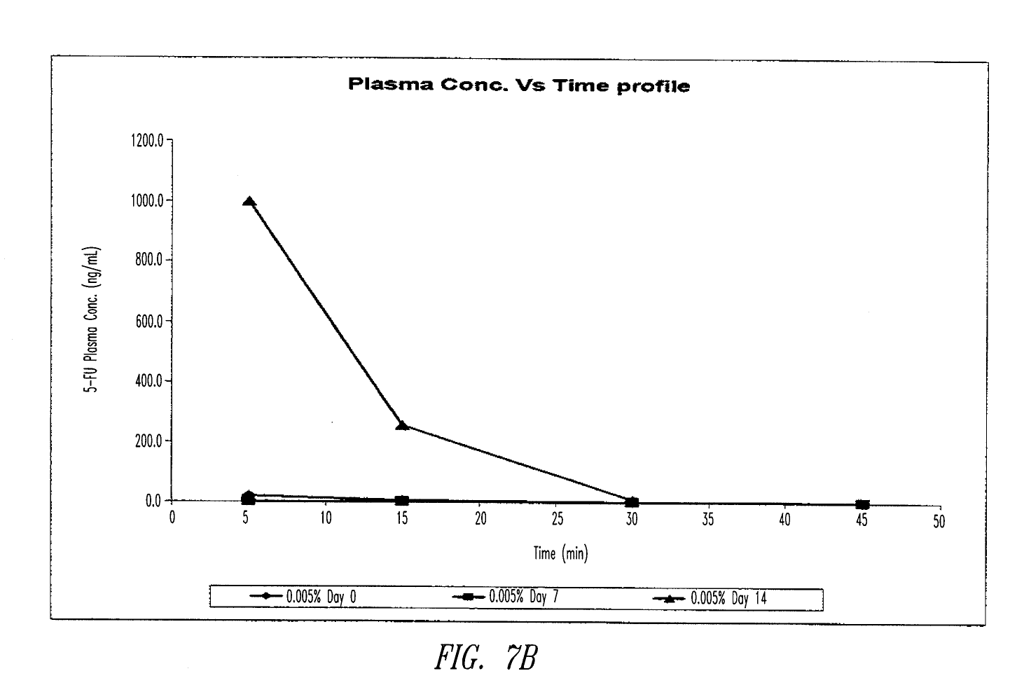

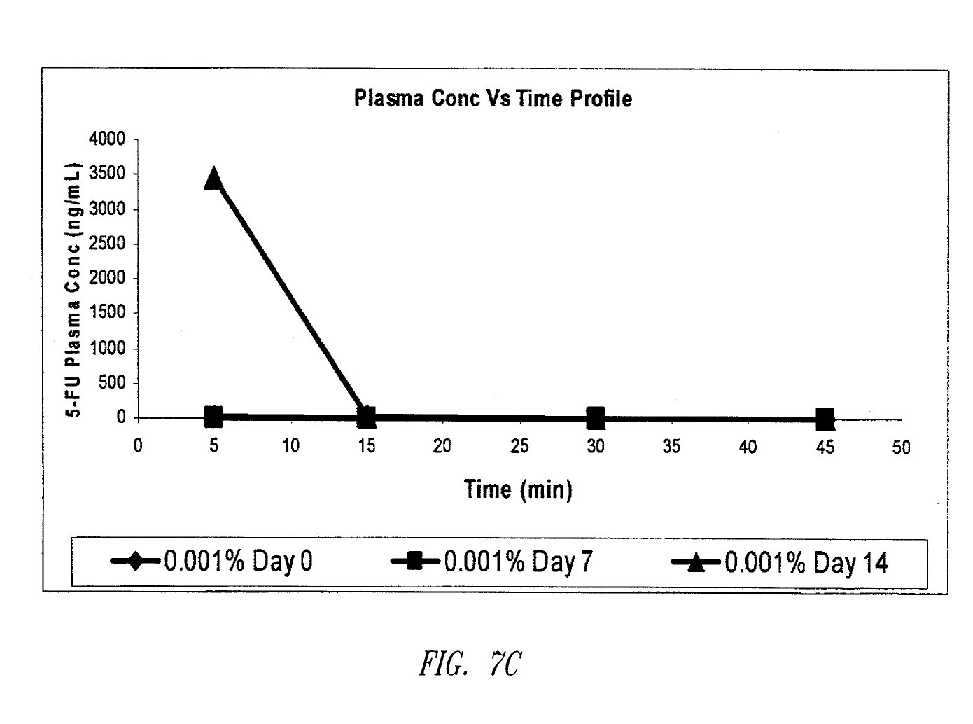

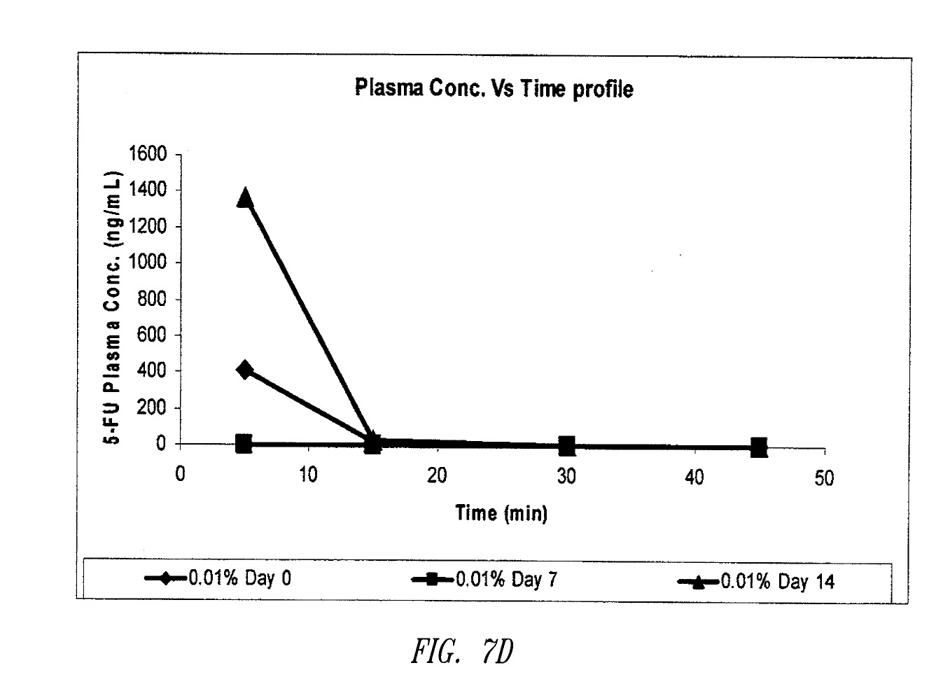

FIG. 7 depicts the pharmacokinetic profile of individual formulations on Day 0, Day 7 and Day 14. It is clear from the profiles that repeated application of topical eniluracil has no effect on 5-FU pharmacokinetics on day 7 but delays plasma 5-FU elimination at 14 days.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A topical formulation for administration to a patient undergoing treatment with 5-FU or a 5-FU prodrug, the formulation comprising an irreversible DPD inhibitor.

2. The formulation of claim 1, wherein the DPD inhibitor comprises a 5-substituted uracil compound or a prodrug thereof.

3. The formulation of claim 1, wherein the DPD inhibitor comprises a uracil compound substituted in the 5-position by a halogen atom, a C₂₋₄ alkenyl group, a C₂₋₄ alkenyl group substituted by halogen, a C₂₋₆ alkynyl group, a C₂₋₆ alkynyl group substituted by a halogen, a cyano group, a C₁₋₄ alkyl group or a C₁₋₄ alkyl group substituted by halogen.

4. The formulation of claim 1, wherein the DPD inhibitor comprises a uracil compound selected from the group consisting of eniluracil, 5-propynyluracil, 5-cyanouracil, 5-propynyluracil, 5-bromoethynyluracil, 5-(1-chlorovinyl)uracil, 5-iodouracil, 5-bromovinyluracil, (E)-5-(2-bromovinyl)uracil 5-hex-1-ynyluracil, 5-vinyluracil, 5-trifluorouracil, 5-bromouracil and 5-(2-bromo-1-chlorovinyl)uracil.

5. A topical formulation for reducing the frequency and/or severity of Hand-Foot Syndrome (HFS) in a patient undergoing treatment with 5-FU or a 5-FU prodrug, the topical formulation comprising an effective dose of an irreversible DPD inhibitor.

6. The topical formulation of claim 5, wherein the effective dose inhibits DPD activity in the hands and/or feet but does not result in systemic DPD inhibition.

7. The topical formulation of claim 5, wherein the concentration of DPD inhibitor in the topical formulation is about 0.0.001 to about 0.05 w/w.

8. The topical formulation of claim 5, wherein the DPD inhibitor comprises a 5-substituted uracil compound or a prodrug thereof.

9. The topical formulation of claim 5, wherein the DPD inhibitor comprises a uracil compound substituted in the 5-position by a halogen atom, a C₂₋₄ alkenyl group, a C₂₋₄ alkenyl group substituted by halogen, a C₂₋₆ alkynyl group, a C₂₋₆ alkynyl group substituted by a halogen, a cyano group, a C₁₋₄ alkyl group or a C₁₋₄ alkyl group substituted by halogen.

10. The topical formulation of claim 5, wherein the DPD inhibitor comprises a uracil compound selected from the group consisting of eniluracil, 5-propynyluracil, 5-cyanouracil, 5-propynyluracil, 5-bromoethynyluracil, 5-(1-chlorovinyl)uracil, 5-iodouracil, 5-bromovinyluracil, (E)-5-(2-bromovinyl)uracil 5-hex-1-ynyluracil, 5-vinyluracil, 5-trifluorouracil, 5-bromouracil and 5-(2-bromo-1-chlorovinyl)uracil.

11. The topical formulation of claim 5, wherein the DPD inhibitor is also an inhibitor of TP and/or UP or wherein the formulation further comprises an inhibitor of TP and/or UP.

12. The topical formulation of claim 5, wherein the DPD inhibitor is eniluracil or a prodrug thereof.

13. The topical formulation of claim 5, wherein the topical formulation is selected from the group consisting of an ointment, cream, lotion, aerosol spray, roll-on liquid and pad form.

14. A method for treating a cancer patient comprising first administering a DPD inhibitor and thereafter administering 5-FU or a 5-FU prodrug, wherein DPD inhibitor is administered as a topical formulation.

15. A method for reducing the frequency and/or severity of Hand-Foot Syndrome (HFS) in a patient undergoing treatment with 5-FU or a 5-FU prodrug, the method comprising applying to the hands and/or feet of said patient a topical formulation comprising an effective dose of an irreversible DPD inhibitor.

16. The method of claim 15, wherein the topical formulation inhibits DPD activity in the hands and/or feet but does not inhibit systemic DPD activity in the patient.

17. The method of claim 15, wherein the concentration of DPD inhibitor in the topical formulation is about 0.0.001 to about 0.05 w/w.

18. The method of claim 15, further comprising the step of removing the topical formulation after an exposure time of about 5 to about 30 minutes.

19. The method of claim 15, wherein the topical formulation is in a form selected from the group consisting of an ointment, cream, lotion, aerosol spray, roll-on liquid and pad form.

20. The method of claim 15, wherein the topical formulation is applied prior to 5-FU or 5-FU prodrug treatment.

21. The method of claim 15, wherein the topical formulation is applied about 5 minutes to about 72 hours prior to 5-FU or 5-FU prodrug treatment.

22. The method of claim 15, wherein the DPD inhibitor comprises a 5-substituted uracil compound or a prodrug thereof.

23. The method of claim 15, wherein the DPD inhibitor comprises a uracil compound substituted in the 5-position by a halogen atom, a C₂₋₄ alkenyl group, a C₂₋₄ alkenyl group substituted by halogen, a C₂₋₆ alkynyl group, a C₂₋₆ alkynyl group substituted by a halogen, a cyano group, a C₁₋₄ alkyl group or a C₁₋₄ alkyl group substituted by halogen.

24. The method of claim 15, wherein the DPD inhibitor comprises a uracil compound selected from the group consisting of eniluracil, 5-propynyluracil, 5-cyanouracil, 5-propynyluracil, 5-bromoethynyluracil, 5-(1-chlorovinyl)uracil, 5-iodouracil, 5-bromovinyluracil, (E)-5-(2-bromovinyl)uracil 5-hex-1-ynyluracil, 5-vinyluracil, 5-trifluorouracil, 5-bromouracil and 5-(2-bromo-1-chlorovinyl)uracil.

25. The method of claim 15, wherein the DPD inhibitor is selected from the group consisting of 5-(phenylselenenyl)uracil (PSU), 5-(phenylthio)uracil (PTU), 5-(phenylselenenyl)barbituric acid and 5-(phenylthio)barbituric acid.

26. The method of claim 15, wherein the DPD inhibitor is also an inhibitor of TP and/or UP or wherein the formulation further comprises an inhibitor of TP and/or UP.

27. The method of claim 15, wherein the DPD inhibitor is eniluracil or a prodrug thereof.

28. The method of claim 15, wherein the 5-FU or 5-FU prodrug is selected from the group and their 5′-esters, including phosphate esters: consisting of 5-fluorouridine, 5-fluorocytidine, 5-fluoro-2-deoxyuridine, 5-fluoro-2-deoxycytidine, 5′-deoxy-4′,5-fluorouridine, and 5-fluoroarabinosyluracil. 5′-Deoxy-5-fluorouridine, 1-(2-tetrahydrofuranyl)-5-fluorouracil, 1-C₁₋₈ alkylcarbamoyl-5-fluorouracil derivative, 1-(2-tetrahydrofuryl)-5-fluorouracil, 5′-deoxy-5-fluoro-N-[(pentyloxy)carbonyl]-cytidine (capecitabine), or a compound that is converted to 5-FU in vivo.

29. The method of claim 15, wherein the 5-FU or 5-FU prodrug is 5-FU or capecitabine.

30. The method of claim 15, wherein the DPD inhibitor is eniluracil and the 5-FU or 5-FU prodrug is 5-FU or capecitabine.
